(19) United States
(12) Patent Application Publication
(10) Pub. No.: US 2026/0295092 A1
(43) Pub. Date: Oct. 1, 2026

(54) DUAL AAV VECTORS FOR TREATING STARGARDT DISEASE

(71) Applicants: University of Florida Research Foundation, Incorporated, Gainesville, FL (US); Atsena Therapeutics, Inc., Durham, NC (US)

(72) Inventors: Sanford L. Boye, Gainesville, FL (US); Shannon E. Boye, Gainesville, FL (US); Kaitlyn Calabro, Cary, NC (US)

(73) Assignees: University of Florida Research Foundation, Incorporated, Gainesville, FL (US); Atsena Therapeutics, Inc., Durham, NC (US)

(21) Appl. No.: 19/480,459

(22) PCT Filed: Apr. 30, 2024
(86) PCT No.: PCT/US2024/027108

§ 371 Date: Oct. 31, 2025

Related U.S. Application Data
(60) Provisional application No. 63/499,482, filed on May 1, 2023.

(51) Int. Cl. A61K 48/00; A61K 9/00; A61P 27/02; C07K 14/47; C12N 5/071; C12N 15/864

(52) U.S. Cl. CPC A61K 48/0058; A61K 9/0019; A61K 48/0075; A61P 27/02; C07K 14/47; C12N 5/0602; C12N 15/8645; C12N 2750/14142; C12N 2750/14143; C12N 2800/40; C12N 2830/34; C12N 2830/50; C12N 2840/44

(57) ABSTRACT

Disclosed herein are compositions and methods for treating diseases of the mammalian eye, and in particular, complications associated with Stargardt Disease. The disclosure provides AAV-based, dual vector systems that facilitate the expression of full-length proteins whose coding sequences exceed that of the polynucleotide packaging capacity of an individual AAV vector. In particular, methods and compositions relating to the expression of full-length ABCA4 using an AAV-based, dual vector system is described herein.

### CROSS REFERENCE

This application claims the benefit of U.S. Provisional Applications No. 63/499,482 filed May 1, 2023, which application is incorporated herein by reference in its entirety.

### SEQUENCE LISTING

The present application is being filed along with a Sequence Listing in electronic format. The Sequence Listing is provided as a file entitled 58774-726.601.xml, created Apr. 25, 2024, which is 93,061 bytes in size. The information in the electronic format of the Sequence Listing is incorporated by reference in its entirety.

### BACKGROUND

Recombinant AAV has emerged as a useful gene delivery vehicle to treat retinal disease. However, one limitation of AAV is its relatively small DNA packaging capacity—approximately 4.7 kilobases (KB). Thus, standard AAV vector systems are unsuitable for addressing diseases in which large genes are mutated or otherwise dysfunctional, such as Stargardt Disease. A solution is needed in order to package large genes into AAV vector systems and safely deliver gene therapy treatment to patients.

### SUMMARY

Disclosed are rAAV dual vector and polynucleotide vector systems and compositions useful in delivering a variety of nucleic acid segments for use in various gene-therapy regimens.

Further disclosed are recombinant viral particles, isolated host cells, and pharmaceutical compositions comprising any of these rAAV dual vector and polynucleotide vector systems. Methods are also provided for preparing and using the improved rAAV dual vector systems disclosed herein in viral-based gene therapies, and in particular, for the treatment and/or amelioration of symptoms of defects in ATP-binding cassette transporter (ABCA4), including, without limitation, the treatment of human Stargardt Disease. In various aspects, the methods of treatment and pharmaceutical compositions provided herein are intended for administration to one or both eyes of a subject, e.g., a human or animal subject.

In some aspects, disclosed herein is a polynucleotide vector system for providing an ABCA4 gene, comprising a first AAV vector polynucleotide comprising a first ABCA4 sequence of the ABCA4 gene, and a second AAV vector polynucleotide comprising a second ABCA4 sequence of the ABCA4 gene, wherein the ABCA4 gene encodes an ABCA4 protein at least 95% identical to SEQ ID NO: 2; the first ABCA4 sequence comprises exon 1 to exon 20 of the ABCA4 gene, and the second ABCA4 sequence comprises exon 21 to exon 50 of the ABCA4 gene. In some embodiments, the last nucleotide of the first ABCA4 sequence and the first nucleotide of the second ABCA4 sequence do not overlap. In some embodiments, the last about 5, 10, 15, 20, 25, 30, 35, 40, 45, or 50 nucleotides of the first ABCA4 sequence and the first about 5, 10, 15, 20, 25, 30, 35, 40, 45, or 50 nucleotides of the second ABCA4 sequence do not overlap. In some embodiments, the first ABCA4 sequence does not comprise any one of exons 21 to exons 50 of the ABCA4 gene, and the second ABCA4 sequence does not comprise any one of exons 1 to exons 20 of the ABCA4 gene. In some embodiments, the ABAC4 gene is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1. In some embodiments, the first ABCA4 sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 13. In some embodiments, the second ABCA4 sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 15. In some embodiments, the first AAV vector polynucleotide comprises a promoter upstream of the first ABCA4 sequence. In some embodiments, the promoter is a smCBA promoter, CMV promoter, EF-1 alpha promoter, cone arrestin promoter, human ABCA4 promoter, TαC gene promoter, rhodopsin promoter, cGMP-phosphodiesterase β-subunit promoter, human rhodopsin promoter, mouse rhodopsin promoter, hGRK1 promoter, rod specific IRBP promoter, or VMD2 promoter. In some embodiments, the promoter is a smCBA promoter. In some embodiments, the promoter comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 23. In some embodiments, the first AAV vector polynucleotide comprises a splice donor site. In some embodiments, the splice donor site is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 24. In some embodiments, the second AAV vector polynucleotide comprises a splice acceptor site. In some embodiments, the splice acceptor site is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 25. In some embodiments, the first AAV vector polynucleotide and/or the second AAV vector polynucleotide comprises an alkaline phosphatase (AP) head sequence. In some embodiments, the first AAV vector polynucleotide comprises the AP head sequence, and the AP head sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 22. In some embodiments, the second AAV vector polynucleotide comprises the AP head sequence, and the AP head sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 22. In some embodiments, the second AAV vector polynucleotide comprises a polyadenylation (pA) signal sequence. In some embodiments, the pA signal sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 21. In some embodiments, the first AAV vector polynucleotide and the second AAV vector polynucleotide each comprise a 5′ AAV ITR and a 3′ AAV ITR. In some embodiments, the 5′ AAV ITR comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 26. In some embodiments, the 3′ AAV ITR comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 27. In some embodiments, the 5′ AAV ITR and the 3′ AAV ITR are of the AAV2 serotype.

In certain aspects, described herein is a recombinant viral particle comprising the first AAV vector polynucleotide or the second AAV vector polynucleotide described herein. In some embodiments, the recombinant viral particle comprises an AAV44.9 (E531D), AAV7m8, AAV-DJ, AAV2/2-MAX, AAVSHh10, AAVSHh10Y, AAV3b, AAVLK03, AAV8BP2, AAV1 (E531K), AAV6 (D532N), AAV6-3pmut, AAV2G9, AAV44.9, AAVrh.8, AAVrh.8R, or AAVAnc80 capsid. In some embodiments, the recombinant viral particle comprises an AAV44.9 (E531D) capsid.

In certain aspects, described herein is an isolated host cell comprising the polynucleotide vector system described herein or the recombinant viral particle described herein. In some embodiments, the cell is a photoreceptor cell, cone cell, rod cell, retinal cell, ganglion cell, retinal pigment epithelium cell, vestibular hair cell, inner ear hair cell, or outer ear hair cell.

In certain aspects, described herein is a method for treating or ameliorating a disease or condition in a human or animal, comprising administering to one or more cells of the human or animal, a polynucleotide vector system described herein or the recombinant viral particle described herein, wherein expression of the ABCA4 gene treats or ameliorates the disease or condition and is expressed in the one or more cells. In some embodiments, the disease or condition is Stargardt Disease. In some embodiments, the treating provides a partial or complete restoration of vision loss. In some embodiments, the polynucleotide vector system is administered by parenteral administration, intravenous administration, intramuscular administration, intraocular administration, intranasal administration, subretinal administration, round window injection, or during cochlear implant surgery.

### BRIEF DESCRIPTION OF DRAWINGS

For promoting an understanding of the principles of the disclosure, reference will now be made to the embodiments, or examples, illustrated in the drawings and specific language will be used to describe the same. It will nevertheless be understood that no limitation of the scope of the disclosure is thereby intended. Any alterations and further modifications in the described embodiments, and any further applications of the principles of the disclosure as described herein are contemplated as would normally occur to one of ordinary skill in the art to which the disclosure relates.

FIG. 1 shows schematics of non-limiting examples of dual vector constructs. The dashed box indicates constructs which have shown efficacy both in vitro and in vivo.

FIG. 2 shows Western Blot results from assessment of dual vector transduction in vitro. The first column (from the left) is a ladder. Columns 2 and 3 are biological replicates (individual transductions) of cultured cells transduced with S007 and S008. Columns 4 and 5 are biological replicates (individual transductions) of cultured cells transduced with S009 and S010. Columns 6 and 7 are biological replicates (individual transductions) of cultured cells transduced with S011 and S012.

FIGS. 3A-3B show confirmation that dual AAV vectors produce robust levels of full length ABCA4 in retinas of Abca4−/− mice. FIG. 3A is an image of Western Blot results showing ABCA4 expression. Starting at the left, column 1 is a ladder. Columns 2-5 are biological replicates (individual mice) transduced with S009 and S0010. Columns 6-9 are biological replicates (individual mice) transduced with S011 and S012. Columns 10-16 are from mice that were not administered vectors. Columns 10-11 are Abca4+/− mice. Column 13 is from Abca4−/− mice. Columns 14-16 are from Abca4+/+ mice. FIG. 3B is a graph showing quantification of ABCA4 expression relative to Wild Type (normalized to Vinculin).

FIG. 4 shows a graph with measurements of image pixel intensity from scanning laser ophthalmoscopy (cSLO) images demonstrating that subretinal injection of S009-S010 ABCA4 dual AAV vectors (SEQ ID NOS: 5 and 6, respectively) is sufficient to lower the retinal autofluorescence phenotype characteristic of Abca4−/− knockout mice compared to vehicle injected contralateral eyes.

FIGS. 5A-5C show graphs of electroretinogram (ERG) data demonstrating that subretinal injection of S009-S010 ABCA4 dual AAV vectors is well tolerated in Abca4−/− mice. ERG responses in dual vector-treated mice are indistinguishable from vehicle injected contralateral eyes at 2-months post injection. FIG. 5A depicts the amplitude of the scotopic a-wave. FIG. 5B depicts the amplitude of the scototopic b-wave. FIG. 5C depicts the amplitude of the phototopic b-wave.

### DETAILED DESCRIPTION

Illustrative embodiments of the disclosure are described below. The disclosure provides materials and methods for genetic therapy of diseases and conditions, such as Stargardt Disease. Stargardt Disease is a form of macular degeneration. The disease is an autosomal recessive disorder which can lead to blindness and which affects 1 in 8,000 people. Stargardt Disease is associated with bi-allelic mutations in the gene encoding the ATP-binding cassette transporter ABCA4. ABCA4 is primarily expressed in photorecepotor cells in the retina. In some embodiments, defects in ABCA4 leads to improper transport of vitamin A. In some embodiments, defects in ABCA4 lead to accumulation of toxic byproducts, such as bisretinoids in the retina.

Aspects of the disclosure concern AAV-based dual vector systems that allow for expression of full-length proteins whose coding sequence exceeds the polynucleotide packaging capacity of individual AAV vectors. The disclosure provides nucleic acid vectors of dual vector systems (e.g., overlap vector systems or hybrid vector systems).

In some embodiments, a vector system of the disclosure employs two discrete AAV vectors that each packages a relatively large DNA molecule (for example, ~4.5 to 4.8 Kb) comprising a portion of an ABCA4 gene. The two vectors are co-administered to selected recipient cells to reconstitute a full-length ABCA4 gene that encodes a biologically-active ABCA4 polypeptide. In some embodiments, a portion of nucleic acid sequence is common to each of the vector genomes (e.g., the common portion contains non-coding sequence). When co-delivered to suitable cells, the common sequence region facilitates the proper concatamerization of the two partial gene cassettes. These gene cassettes then undergo homologous recombination to produce a full-length gene cassette within the cells. The common nucleic acid sequence is then spliced out from the resulting RNA. Non-limiting components of non-limiting embodiments of the dual vector systems include the use of AAV inverted terminal repeats (ITR), the small (truncated) version of the chimeric CMV/chicken β-actin promoter (smCBA), human ABCA4 cDNA sequence and the mini bovine growth hormone polyadenylation (pA) signal (mini2 pA).

### Dual Vector Systems

In some aspects of the disclosure, a dual vector system of the disclosure includes:
(i) a first AAV vector polynucleotide comprising an inverted terminal repeat at each end (for example, the 5′-end and the 3′-end) of the polynucleotide, and between the inverted terminal repeats a suitable promoter followed by (for example, 3′ to the promoter) a partial coding sequence that encodes an N-terminal part of a selected full-length polypeptide followed by a splice donor site and an intron, and
(ii) a second AAV vector polynucleotide comprising an inverted terminal repeat at each end (5′-end and 3′-end) of the polynucleotide, and between the inverted terminal repeats an intron and a splice acceptor site for the intron, optionally followed by a partial coding sequence that encodes a C-terminal part of the selected full-length polypeptide, optionally followed by a polyadenylation (pA) signal sequence. In some embodiments, the intron sequence in the first and second AAV vectors comprises an alkaline phosphatase homologous recombination sequence (APhead), e.g., a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 22.

In some embodiments, the split point between the first and second AAV polynucleotide sequences is between exon 19 and exon 20 of the hABCA4 gene. In some embodiments, the split point between the first and second AAV polynucleotide sequences is between exon 20 and exon 21 of the hABCA4 gene. In some embodiments, the split point between the first and second AAV vector polynucleotide sequences is between exon 21 and exon 22 of the hABCA4 gene.

In some embodiments, the first AAV vector polynucleotide comprises the nucleotide sequence of SEQ ID NO: 3, 5, or 7, or a functional fragment and/or variant thereof, and the second AAV vector polynucleotide comprises the nucleotide sequence of SEQ ID NO: 4, 6, or 8, or a functional fragment and/or variant thereof. In some embodiments, the first AAV vector polynucleotide comprises the nucleotide sequence of SEQ ID NO: 3, 5, or 7, or a sequence 85%, 86%, 87%, 88%, 89%, 90%, 91%, 92%, 93%, 94%, 95%, 96%, 97%, 98%, or 99% identical thereto, and the second AAV vector polynucleotide comprises the nucleotide sequence of SEQ ID NO: 4, 6, or 8, a sequence 85%, 86%, 87%, 88%, 89%, 90%, 91%, 92%, 93%, 94%, 95%, 96%, 97%, 98%, or 99% identical thereto. In some embodiments, the first AAV vector polynucleotide comprises the nucleotide sequence of SEQ ID NO: 3, or a sequence 85%, 86%, 87%, 88%, 89%, 90%, 91%, 92%, 93%, 94%, 95%, 96%, 97%, 98%, or 99% identical thereto, and the second AAV vector polynucleotide comprises the nucleotide sequence of SEQ ID NO: 4, a sequence 85%, 86%, 87%, 88%, 89%, 90%, 91%, 92%, 93%, 94%, 95%, 96%, 97%, 98%, or 99% identical thereto. In some embodiments, the first AAV vector polynucleotide comprises the nucleotide sequence of SEQ ID NO: 5, or a sequence 85%, 86%, 87%, 88%, 89%, 90%, 91%, 92%, 93%, 94%, 95%, 96%, 97%, 98%, or 99% identical thereto, and the second AAV vector polynucleotide comprises the nucleotide sequence of SEQ ID NO: 6, a sequence 85%, 86%, 87%, 88%, 89%, 90%, 91%, 92%, 93%, 94%, 95%, 96%, 97%, 98%, or 99% identical thereto. In some embodiments, the first AAV vector polynucleotide comprises the nucleotide sequence of SEQ ID NO: 7, or a sequence 85%, 86%, 87%, 88%, 89%, 90%, 91%, 92%, 93%, 94%, 95%, 96%, 97%, 98%, or 99% identical thereto, and the second AAV vector polynucleotide comprises the nucleotide sequence of SEQ ID NO: 8, a sequence 85%, 86%, 87%, 88%, 89%, 90%, 91%, 92%, 93%, 94%, 95%, 96%, 97%, 98%, or 99% identical thereto.

The coding sequences in the first and second vectors when combined encode the selected full-length polypeptide, or a functional fragment or variant thereof. In some embodiments, the selected full-length polypeptide is human ABCA4 or hABCA4.

In some embodiments, all or part of the intron sequence present at the 3′-end of the coding sequence of the first vector is identical or substantially identical with all or part of the intron sequence present at the 5′-end of the coding sequence of the second vector.

In particular embodiments, the intron sequence utilized in any vector system of the disclosure is a sequence of an intron naturally present in the genomic sequence of the gene encoding the selected polypeptide. In some embodiments, the intron comprises an alkaline phosphatase (AP) sequence. In some embodiments, the intron comprises an alkaline phosphatase homologous recombination sequence (APhead). In some embodiments, the alkaline phosphatase homologous recombination sequence comprises the nucleotide sequence of SEQ ID NO: 22, or a sequence 85%, 86%, 87%, 88%, 89%, 90%, 91%, 92%, 93%, 94%, 95%, 96%, 97%, 98%, or 99% identical thereto.

Example strategies that may overcome the issue of random concatemerization and thereby increase specificity as well as efficiency of these dual vector platforms are provided herein. First, the addition of a highly-recombinogenic sequence such as that used in example dual vectors here results in significantly increased protein expression compared with the trans-splicing system. The finding that AP dual vectors are more efficient than trans-splicing vectors supports that the AP sequence directs at least some of the concatemerization events toward the proper orientation with recombination then occurring via this sequence or via the ITRs. The APhead domain in particular can mediate appropriate head-to-tail concatemerization following re-combination of the dual vectors in the cell. In some embodiments, example dual vector systems having an AP sequence facilities proper alignment of more concatemers. In some embodiments, example dual vector systems having an AP sequence mediates a more-efficient expression of ABCA4. In some embodiments, dual vector systems without AP sequences are used.

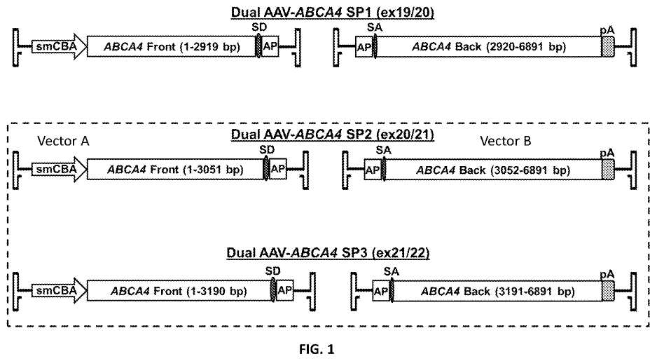

In some embodiments, the intron sequence utilized in the dual vector system of the disclosure is a sequence of an intron that is not naturally present in the genomic sequence of a gene encoding the selected polypeptide. In some embodiments, the intron sequence is derived from the MYO7A gene. In particular embodiments, the intron is a synthetic alkaline phosphatase (AP) intron. The intron sequences utilized in the dual vector system of the disclosure can comprise splice donor and splice acceptor sequences. In some embodiments, the intron sequence is a recombinogenic, intronic sequence (for example, the AK sequence of the F1 phage). In these embodiments, the dual vectors rely on both ITR-mediated concatemerization and homologous recombination mediated by the AK sequence for the reconstitution of the full-length expression cassette. Thus, in some embodiments, the intron sequence is the AK sequence of the F1 phage. Accordingly, in some embodiments of the disclosed dual vectors, the vectors comprise one or more AP intronic spliceosome recognition sites, such as one or more AP splice acceptor (APSA) domains or AP splice donor (APSD) domains. In some embodiments, these vectors comprise an APSA and an APSD. In some embodiments, the front half vector contains an APSA and the back half vector contains an APSD. In some embodiments, the front half vector contains an APSD and the back half vector contains an APSA. See FIG. 1. In some embodiments, the vectors comprise one or more non-AP intronic spliceosome recognition sites. In some embodiments, the splice donor comprises the nucleotide sequence of SEQ ID NO: 24, or a sequence 85%, 86%, 87%, 88%, 89%, 90%, 91%, 92%, 93%, 94%, 95%, 96%, 97%, 98%, or 99% identical thereto. In some embodiments, the splice acceptor comprises the nucleotide sequence of SEQ ID NO: 25, or a sequence 85%, 86%, 87%, 88%, 89%, 90%, 91%, 92%, 93%, 94%, 95%, 96%, 97%, 98%, or 99% identical thereto.

Another approach for directing concatemerization is the use of single-strand oligonucleotides that are capable of tethering the back end of the 5′ vector and the front end of the 3′ vector together. However, this strategy requires efficient delivery of the oligonucleotide to the nucleus of the target cells timed with the dual vectors. Finally, dual vectors utilizing mismatched ITRs can be used to direct concatemerization in a head-to-tail orientation, although the process may require further optimization of the AAV packaging machinery. Accordingly, in some embodiments, the dual vector pairs contain an APhead-encoding sequence as part of the AP intron.

Thus, in some embodiments, the first AAV vector polynucleotide comprises the nucleotide sequence of SEQ ID NO: 3, 5, or 7, or a functional fragment and/or variant thereof, and the second AAV vector polynucleotide comprises the nucleotide sequence of SEQ ID NO: 4, 6, or 8, or a functional fragment and/or variant thereof. In some embodiments, the intronic sequence is the AK sequence of the F1 phage.

In some embodiments, the split point between the first and second AAV vector polynucleotide sequences is between exon 21 and exon 22 of the hABCA4 gene. In some embodiments, the split point between the first and second AAV vector polynucleotide sequences is between exon 20 and exon 21 of the hABCA4 gene. In some embodiments, the split point between the first and the second AAV vector polynucleotide sequence is between nucleic acid 3050 and 3051 of the hABCA4 gene, as numbered in SEQ ID NO: 1. In some embodiments, the split point between the first and second AAV vector polynucleotide sequences is between exon 19 and exon 20 of the hABCA4 gene.

In some embodiments, the first AAV vector polynucleotide comprises a nucleotide sequence encoding an N-terminal portion of ABCA4. In some embodiments, the first AAV vector polynucleotide comprises the sequence of SEQ ID NO: 9, SEQ ID NO: 13 or SEQ ID NO: 17, or a sequence 85%, 86%, 87%, 88%, 89%, 90%, 91%, 92%, 93%, 94%, 95%, 96%, 97%, 98% or 99% identical thereto. In some embodiments, the first AAV vector encodes an N-terminal portion of ABCA4 comprising the sequence of SEQ ID NO: 10, SEQ ID NO: 14 or SEQ ID NO: 18, or a sequence 85%, 86%, 87%, 88%, 89%, 90%, 91%, 92%, 93%, 94%, 95%, 96%, 97%, 98% or 99% identical thereto. In some embodiments, the first AAV vector polynucleotide comprises the sequence of SEQ ID NO: 9 or a sequence 85%, 86%, 87%, 88%, 89%, 90%, 91%, 92%, 93%, 94%, 95%, 96%, 97%, 98% or 99% identical thereto. In some embodiments, the first AAV vector encodes an N-terminal portion of ABCA4 comprising the sequence of SEQ ID NO: 10, or a sequence 85%, 86%, 87%, 88%, 89%, 90%, 91%, 92%, 93%, 94%, 95%, 96%, 97%, 98% or 99% identical thereto. In some embodiments, the first AAV vector polynucleotide comprises the sequence of SEQ ID NO: 13, or a sequence 85%, 86%, 87%, 88%, 89%, 90%, 91%, 92%, 93%, 94%, 95%, 96%, 97%, 98% or 99% identical thereto. In some embodiments, the first AAV vector encodes an N-terminal portion of ABCA4 comprising the sequence of SEQ ID NO: 14, or a sequence 85%, 86%, 87%, 88%, 89%, 90%, 91%, 92%, 93%, 94%, 95%, 96%, 97%, 98% or 99% identical thereto. In some embodiments, the first AAV vector polynucleotide comprises the sequence of SEQ ID NO: 17, or a sequence 85%, 86%, 87%, 88%, 89%, 90%, 91%, 92%, 93%, 94%, 95%, 96%, 97%, 98% or 99% identical thereto. In some embodiments, the first AAV vector encodes an N-terminal portion of ABCA4 comprising the sequence of SEQ ID NO: 18, or a sequence 85%, 86%, 87%, 88%, 89%, 90%, 91%, 92%, 93%, 94%, 95%, 96%, 97%, 98% or 99% identical thereto.

In some embodiments, the second AAV vector polynucleotide comprises a nucleotide sequence encoding a C-terminal portion of ABCA4. In some embodiments, the second AAV vector polynucleotide comprises the sequence of SEQ ID NO: 11, SEQ ID NO: 15 or SEQ ID NO: 19, or a sequence 85%, 86%, 87%, 88%, 89%, 90%, 91%, 92%, 93%, 94%, 95%, 96%, 97%, 98% or 99% identical thereto. In some embodiments, the second AAV vector encodes a C-terminal portion of ABCA4 comprising the sequence of SEQ ID NO: 12, SEQ ID NO: 16, or SEQ ID NO: 20, or a sequence 85%, 86%, 87%, 88%, 89%, 90%, 91%, 92%, 93%, 94%, 95%, 96%, 97%, 98% or 99% identical thereto. In some embodiments, the second AAV vector polynucleotide comprises the sequence of SEQ ID NO: 11, or a sequence 85%, 86%, 87%, 88%, 89%, 90%, 91%, 92%, 93%, 94%, 95%, 96%, 97%, 98% or 99% identical thereto. In some embodiments, the second AAV vector encodes a C-terminal portion of ABCA4 comprising the sequence of SEQ ID NO: 12, or a sequence 85%, 86%, 87%, 88%, 89%, 90%, 91%, 92%, 93%, 94%, 95%, 96%, 97%, 98% or 99% identical thereto. In some embodiments, the second AAV vector polynucleotide comprises the sequence of SEQ ID NO: 15, or a sequence 85%, 86%, 87%, 88%, 89%, 90%, 91%, 92%, 93%, 94%, 95%, 96%, 97%, 98% or 99% identical thereto. In some embodiments, the second AAV vector encodes a C-terminal portion of ABCA4 comprising the sequence of SEQ ID NO: 16, or a sequence 85%, 86%, 87%, 88%, 89%, 90%, 91%, 92%, 93%, 94%, 95%, 96%, 97%, 98% or 99% identical thereto. In some embodiments, the second AAV vector polynucleotide comprises the sequence of SEQ ID NO: 19, or a sequence 85%, 86%, 87%, 88%, 89%, 90%, 91%, 92%, 93%, 94%, 95%, 96%, 97%, 98% or 99% identical thereto. In some embodiments, the second AAV vector encodes a C-terminal portion of ABCA4 comprising the sequence of SEQ ID NO: 20, or a sequence 85%, 86%, 87%, 88%, 89%, 90%, 91%, 92%, 93%, 94%, 95%, 96%, 97%, 98% or 99% identical thereto.

In some embodiments, polynucleotide vector systems are provided that comprise:
i) a first AAV vector polynucleotide comprising an inverted terminal repeat at each end of the polynucleotide, and between the inverted terminal repeats a promoter followed by a partial coding sequence that encodes an N-terminal part of an ABCA4 polypeptide followed by a splice donor site and an intron, and
ii) a second AAV vector polynucleotide comprising an inverted terminal repeat at each end of the polynucleotide, and between the inverted terminal repeats an intron and a splice acceptor site for the intron. In some embodiments, between the inverted terminal repeats is an intron and a splice acceptor site for the intron, followed by a partial coding sequence that encodes a C-terminal part of the ABCA4 polypeptide, optionally followed by a polyadenylation (pA) signal sequence.

In some embodiments, the intron sequence in the first and second AAV vectors comprises a common polynucleotide sequence, where the common polynucleotide sequence is not part of the coding sequence that encodes the ABCA4 polypeptide. In some embodiments, the intron sequence in the first AAV vector and the intron sequence in the second AAV vector comprise a sequence at least 50%, 60%, 70%, 80%, 85%, 86%, 87%, 88%, 89%, 90%, 91%, 92%, 93%, 94%, 95%, 96%, 97%, 98%, 99%, or 100% identical to SEQ ID NO: 22.

In some embodiments, the inverted terminal repeat comprises the sequence of SEQ ID NO: 26 or 27, or a sequence 85%, 86%, 87%, 88%, 89%, 90%, 91%, 92%, 93%, 94%, 95%, 96%, 97%, 98% or 99% identical thereto. In some embodiments, the promotor comprises the sequence of SEQ ID NO: 23, or a sequence 85%, 86%, 87%, 88%, 89%, 90%, 91%, 92%, 93%, 94%, 95%, 96%, 97%, 98% or 99% identical thereto. In some embodiments, the N-terminal part of the ABCA4 polypeptide comprises the sequence of SEQ ID NO: 14, or a sequence 85%, 86%, 87%, 88%, 89%, 90%, 91%, 92%, 93%, 94%, 95%, 96%, 97%, 98% or 99% identical thereto. In some embodiments, the N-terminal part of the ABCA4 polypeptide is encoded by a sequence comprising the sequence of SEQ ID NO: 13, or a sequence 85%, 86%, 87%, 88%, 89%, 90%, 91%, 92%, 93%, 94%, 95%, 96%, 97%, 98% or 99% identical thereto. In some embodiments, the C-terminal part of the ABCA4 polypeptide comprises the sequence of SEQ ID NO: 16, or a sequence 85%, 86%, 87%, 88%, 89%, 90%, 91%, 92%, 93%, 94%, 95%, 96%, 97%, 98% or 99% identical thereto. In some embodiments, the C-terminal part of the ABCA4 polypeptide is encoded by a sequence comprising the sequence of SEQ ID NO: 15, or a sequence 85%, 86%, 87%, 88%, 89%, 90%, 91%, 92%, 93%, 94%, 95%, 96%, 97%, 98% or 99% identical thereto. In some embodiments, the splice donor site comprises the sequence of SEQ ID NO: 24, or a sequence 85%, 86%, 87%, 88%, 89%, 90%, 91%, 92%, 93%, 94%, 95%, 96%, 97%, 98% or 99% identical thereto. In some embodiments, the splice acceptor site comprises the sequence of SEQ ID NO: 25, or a sequence 85%, 86%, 87%, 88%, 89%, 90%, 91%, 92%, 93%, 94%, 95%, 96%, 97%, 98% or 99% identical thereto. In some embodiments, the intron comprises the sequence of SEQ ID NO: 22, or a sequence 85%, 86%, 87%, 88%, 89%, 90%, 91%, 92%, 93%, 94%, 95%, 96%, 97%, 98% or 99% identical thereto.

In some embodiments of the dual vector systems described herein, the selected full-length polypeptide is an ABC transporter polypeptide. In some embodiments, the ABC transporter is a human ABCA polypeptide. In some embodiments, the ABCA polypeptide is ABCA4. In some embodiments, full-length ABCA4 is encoded in the provided vector systems.

The coding sequences in the first and second vectors when combined encode the selected full-length polypeptide, or a functional fragment or variant thereof. Accordingly, in some embodiments, all or part of the intron sequence present at the 3′-end of the coding sequence of the first vector is identical or substantially identical with all or part of the intron sequence present at the 5′-end of the coding sequence of the second vector, where the intron sequence is not part of the coding sequence that encodes the ABCA4 polypeptide.

Some embodiments of the dual vectors described herein contemplate a virus or a recombinant viral particle comprising the first AAV vector polynucleotide or the second AAV vector polynucleotide as described herein. In particular embodiments, the first AAV vector polynucleotide comprises a sequence at least 85%, 86%, 87%, 88%, 89%, 90%, 91%, 92%, 93%, 94%, 95%, 96%, 97%, 98% or 99% identical to SEQ ID NO: 5, and the second AAV vector polynucleotide comprises a sequence at least 85%, 86%, 87%, 88%, 89%, 90%, 91%, 92%, 93%, 94%, 95%, 96%, 97%, 98% or 99% identical to SEQ ID NO: 6. In some embodiments, the virus or recombinant viral particle is characterized as an adeno-associated virus (AAV) or an infectious AAV viral particle. In some embodiments, the recombinant AAV viral particle includes one or more tyrosine-to-phenylalanine (Y-F) mutations in a capsid protein of the virus or virion. Tyrosine-to-phenylalanine (Y-F) mutations in a capsid protein of the virus or virion at amino acid position 733 are specifically contemplated herein (for example, AAV8 Y733F). Likewise, tyrosine-to-phenylalanine (Y-F) mutations in a capsid protein of the virus or virion at amino acid position 731 are specifically contemplated herein (for example, AAV44.9 (Y731F)).

In some embodiments, the virus or virion is packaged in an AAV5, AAV7, AAV8, AAV9, AAV44.9, AAV44.9 (E531D), AAV2 (4pMut) AHS, AAV2, AAVAnc80, AAVrh.8, AAVrh.8R, AAVrh.10, or AAVrh.74 capsid. In some embodiments, the viral particle comprises an AAV1, AAV2, AAV3, AAV4, AAV5, AAV6, AAV7, AAV8, AAV9, AAV10, AAV7m8, AAV-DJ, AAV2/2-MAX, AAVSHh10, AAVSHh10Y, AAV3b, AAVLK03, AAV8PB2, AAV1 (E531K), AAV6 (D532N), AAV6-3pmut, AAV2G9, AAV44.9, AAV44.9 (E531D), AAVrh.8, AAVrh.8R, and/or AAVAnc80 capsid. In some embodiments, the virion is packaged in an AAV44.9 (E531D) capsid variant.

In some embodiments, the dual polynucleotide vector systems described herein use a tissue-specific promoter. In some embodiments, the systems use a promoter that mediates expression in the eye.

In some embodiments, the dual polynucleotide vector systems described herein uses any one of the following promoters: a cytomegalovirus (CMV) promoter, an elongation factor-1 alpha (EF-1 alpha) promoter, a cone arrestin promoter, a chimeric CMV β actin (smCBA) promoter, an ABCA4 gene-derived promoter, a cone transducin a (TαC) gene-derived promoter, a rhodopsin promoter, a cGMP-phosphodiesterase β-subunit promoter, human or mouse rhodopsin promoter, a human rhodopsin kinase (hGRK1) promoter, a rod specific IRBP promoter, a RPE-specific vitelliform macular dystrophy-2 [VMD2] promoter, and combinations thereof. In some embodiments, the polynucleotide vector system described herein uses a human rhodopsin kinase (hGRK1) promoter. In some embodiments, the polynucleotide vector system uses a cone arrestin promoter. In some embodiments, the polynucleotide vector system uses a cytomegalovirus (CMV) promoter. In some embodiments, the polynucleotide vector system uses an elongation factor-1 alpha (EF-1 alpha) promoter.

In some embodiments, any vector of the dual polynucleotide vector systems described in the disclosure may be administered by parenteral administration, such as intravenous, intramuscular, intraocular, intranasal, etc. The vector can be administered in vivo, in vitro or ex vivo. In particular embodiments, a vector provided herein may be administered by subretinal injection. The vector may be administered in vivo or ex vivo.

In some embodiments, any vector of the dual polynucleotide vector systems described herein may be administered to the eye. In particular embodiments, a vector is administered to the eye of a subject by subretinal injection.

The methods of the disclosure can be used with humans and other animals. Animals contemplated within the scope of the disclosure include, for example, dogs, cats, rabbits, ferrets, guinea pigs, hamsters, pigs, monkeys or other primates, mice, gerbils, horses, mules, donkeys, burros, cattle, cows, pigs, sheep, and alligators. As used herein, the terms “patient” and “subject” are used interchangeably and are intended to include such human and non-human species, including human and non-human cells. Likewise, in vitro methods of the disclosure may also be performed on cells of one or more human or non-human, mammalian species, including human and non-human cells.

### Components of Non-limiting Examples of Dual AAV Vectors

Any of the dual polynucleotide vector systems of the disclosure may be used in conjunction with an AAV vector system known in the art. In treating some diseases, it may be preferable to administer the rAAV vector construct a single time, while in the management or treatment of other diseases or conditions, it may be desirable to provide two or more administrations of the vector constructs to the patient in one or more administration periods. In such circumstances, the AAV vector-based therapeutics may be provided successively in one or more daily, weekly, monthly, or less-frequent periods, as may be necessary to achieve treatment, or amelioration of one or more symptoms of the disease or disorder being treated. In some embodiments, the vector may be provided to one or both eyes by one or more administrations of an infectious adeno-associated viral particle, an rAAV virion, or a plurality of infectious rAAV particles in an amount and for a time sufficient to treat or ameliorate one or more symptoms of the disease or condition being treated.

Some aspects of the disclosed vectors contemplate a virus or a recombinant viral particle comprising the first AAV vector polynucleotide or the second AAV vector polynucleotide as described herein. In some embodiments, the virus or recombinant viral particle is characterized as an adeno-associated virus (AAV) or an infectious AAV viral particle. In some embodiments, the recombinant AAV viral particle includes one or more tyrosine-to-phenylalanine (Y-F) mutations in a capsid protein of the virus or virion. Tyrosine-to-phenylalanine (Y-F) mutations in a capsid protein of the virus or virion at amino acid position 733 are specifically contemplated herein (for example, AAV8 Y733F).

In some embodiments, the virus or virion is packaged in an AAV5, AAV7, AAV8, AAV9, AAV44.9, AAV44.9 (E531D), AAV2 (4pMut) AHS, AAV2, AAVAnc80, AAVrh.8, AAVrh.8R, AAVrh.10, or AAVrh.74 capsid. In some embodiments, the viral particle comprises an AAV1, AAV2, AAV3, AAV4, AAV5, AAV6, AAV7, AAV8, AAV9, AAV10, AAV7m8, AAV-DJ, AAV2/2-MAX, AAVSHh10, AAVSHh10Y, AAV3b, AAVLK03, AAV8PB2, AAV1 (E531K), AAV6 (D532N), AAV6-3pmut, AAV2G9, AAV44.9, AAV44.9 (E531D), AAVrh.8, AAVrh.8R, and/or AAVAnc80 capsid. In some embodiments, the virion is packaged in an AAV44.9 (E531D) capsid variant.

In some embodiments, the polynucleotide vector systems described herein use a tissue-specific promoter. In some embodiments, the systems use a promoter that mediates expression in the eye.

In some embodiments, the polynucleotide vector systems described herein uses any one of the following promoters: a cytomegalovirus (CMV) promoter, an elongation factor-1 alpha (EF-1 alpha) promoter, a cone arrestin promoter, a chimeric CMV β actin promoter (CBA), a truncated chimeric CMV β actin (smCBA) promoter, a human ABCA4 gene-derived promoter, a cone transducin a (TαC) gene-derived promoter, a rhodopsin promoter, a cGMP-phosphodiesterase β-subunit promoter, human or mouse rhodopsin promoter, a human rhodopsin kinase (hGRK1) promoter, a rod specific IRBP promoter, a RPE-specific vitelliform macular dystrophy-2 [VMD2] promoter, and combinations thereof. In some embodiments, the polynucleotide vector system described herein uses a human rhodopsin kinase (hGRK1) promoter. In some embodiments, the polynucleotide vector system uses a cone arrestin promoter. In some embodiments, the polynucleotide vector system uses a cytomegalovirus (CMV) promoter. In some embodiments, the polynucleotide vector system uses an elongation factor-1 alpha (EF-1 alpha) promoter.

In particular embodiments, the disclosure provides rAAV particles been derived from a number of different serotypes, including, for example, those selected from the group consisting of AAV1, AAV2, AAV3, AAV4, AAV5, AAV6, AAV7, AAV8, AAV9, and AAV10. In some embodiments, particles derived from AAV2, AAV5 and AAV8 serotype vectors are utilized. In particular embodiments, particles having an AAV8 (Y733F) or AAV2 (tripleY-F) capsid are used. Accordingly, the disclosure provides recombinant AAV particles derived from, e.g., AAV8 (Y733F) or AAV2 (tripleY-F), that comprise dual polynucleotide vector systems. In some embodiments, the serotype of the AAV vector is not AAV6 or AAV2.

Additional non-limiting capsids include AAV2, AAV6, and capsids derived from AAV2 and AAV6. Such capsids include AAV7m8, AAV-DJ, AAV2/2-MAX, AAVSHh10, AAVSHh10Y, AAV3b, AAVLK03, AAV8PB2, AAV1 (E531K), AAV6 (D532N), AAV6-3pmut, AAV2G9, AAV2G9.AAV44.9, AAV44.9 (E531D), AAVrh.8, AAVrh.8R, and/or AAVAnc80. In some embodiments, the virus or virion is packaged in an AAV5, AAV7, AAV8, AAV9, AAV44.9, AAV44.9 (E531D), AAV2 (4pMut) AHS, AAV2, AAVAnc80, AAVrh.8, AAVrh.8R, AAVrh.10, or AAVrh.74 capsid.

The AAV2/2-MAX capsid comprises five point mutations, Y272F, Y444F, Y500F, Y730F, T491V. The AAVSHh10 and AAV6 (D532N) capsids are both derivatives of AAV6

Additional capsids suitable for use with the disclosed methods include the following: capsids comprising non-native amino acid substitutions at amino acid residues of a wild-type AAV2 capsid, wherein the non-native amino acid substitutions comprise one or more of Y272F, Y444F, T491V, Y500F, Y700F, Y704F and Y730F; capsids comprising non-native amino acid substitutions at amino acid residues of a wild-type AAV6 capsid, wherein the non-native amino acid substitutions comprise one or more of Y445F, Y705F, Y731F, T492V and S663V. In certain embodiments, the capsid comprises AAV2G9, a variant of AAV2.

In other embodiments, the capsid comprises a non-native amino acid substitution at amino acid residue 533 or 733 of a wild-type AAV8 capsid, wherein the non-native amino acid substitution is E533K, Y733F, or a combination thereof. In certain embodiments of the disclosed methods, the capsid comprises AAV8PB2, a variant of AAV8.

In other embodiments, the capsid comprises non-native amino acid substitutions of a wild-type AAV2 capsid comprising one or more of the following mutations:

$$Y 444 F ; \qquad ( a )$$

$$Y 444 F + Y 500 F + Y 730 F ; \qquad ( b )$$

$$Y 272 F + Y 444 F + Y 500 F + Y 730 F ; \qquad ( c )$$

$$Y 444 F + Y 500 F + Y 730 F + T 491 V ; or \qquad ( d )$$

$$Y 272 F + Y 444 F + Y 500 F + Y 730 F + T 491 V . \qquad ( e )$$

In other embodiments, the capsid comprises non-native amino acid substitutions of a wild-type AAV6 capsid, comprising one or more of the following mutations:

$$Y 445 F ; \qquad ( a )$$

$$Y 705 F + Y 731 F ; \qquad ( b )$$

$$T 492 V ; \qquad ( c )$$

$$Y 705 F + Y 731 F + T 492 V ; \qquad ( d )$$

$$S 663 V ; or \qquad ( e )$$

$$S 663 V + T 492 V . \qquad ( f )$$

Additional capsids suitable for use with the disclosed methods are described in International Patent Publication No. WO 2018/156654, published Aug. 30, 2018, herein incorporated by reference in its entirety. In particular embodiments, the rAAV particles disclosed herein comprise one of the following capsids: DGE-DF (also known as ‘V1V4 VR-V’), P2-V2, P2-V3, P2-V1 (also known as ME-B), and P2-V1 (Y-F+T-V) (also known as and ME-B(Y-F+T-V)). In still other embodiments, the rAAV particles may comprise a capsid selected from AAV6 (3pMut) or AAV2 (quadYF+T-V). In still other embodiments, the rAAV particles of the disclosed methods may comprise any of the capsid variants described in International Patent Publication No. WO 2018/156654.

In particular embodiments, disclosed herein are rAAV particles which may comprise a DGE-DF capsid, P2-V2 capsid, P2-V3 capsid, P2-V1 capsid (also known as ME-B), or P2-V1 (Y-F+T-V) or ME-B(Y-F+T-V) capsid for the enhanced transduction of said rAAV particles in retinal cells. In other embodiments, the disclosed rAAV particles may comprise a capsid selected from AAV2 (Y444F), AAV2 (Y444F+Y500F+Y730F), AAV2 (Y272F+Y444F+Y500F+Y730F), AAV2 (Y444F+Y500F+Y730F+T491V) and AAV2 (Y272F+Y444F+Y500F+Y730F+T491V), AAV6 (Y445F), AAV6 (Y705F+Y731F), AAV6 (Y705F+Y731F+T492V), AAV6 (S663V), AAV6 (T492V) or AAV6 (S663V+T492V).

Inverted terminal repeat (ITR) sequences used in any AAV vector systems of the disclosure may comprise any AAV ITR. In some embodiments, the ITRs used in an AAV vector are the same. In some embodiments, the ITRs used in an AAV vector are different. In some embodiments, the ITR may be obtained from an AAV serotype 2 (AAV2), AAV serotype 5 (AAV5), AAV serotype 7 (AAV7), AAV serotype 8 (AAV8), AAV serotype 44.9 (AAV44.9), or a variant thereof, such as AAV serotype 44.9 (E531D) and 44.9 (Y7331F) (see PCT Application No. PCT/US2020/14838, filed Jan. 23, 2020, herein incorporated by reference). In some embodiments, an AAV vector of the disclosure comprises different AAV ITRs. In a non-limiting example, a vector may comprise an ITR of AAV2 and an ITR of AAV5. AAV ITR sequences are well known in the art (see, e.g., GenBank Accession Nos. AF043303.1; NC_001401.2; J01901.1; JN898962.1; K01624.1; and K01625.1). In some embodiments, AAV dual vector systems disclosed herein are able to efficiently express a therapeutic gene that is larger than what may ordinarily be packaged within a single AAV vector.

Accordingly, in some aspects the disclosure provides a virus or virion comprising any of the polynucleotides or vectors of the disclosure. In some embodiments, the virus or virion is an AAV virus. Methods for preparing viruses and virions comprising a heterologous polynucleotide or vector are known in the art. In the case of AAV, cells can be co-infected or transfected with adenovirus or polynucleotide vectors comprising adenovirus genes suitable for AAV helper function.

In some embodiments, the AAV serotype provides for one or more tyrosine to phenylalanine (Y-F) mutations on the capsid surface. In some embodiments, the AAV is an AAV8 serotype having a tyrosine-to-phenylalanine (Y-F) mutation at position 733 (Y733F).

In some embodiments, a triple-mutant AAV8 vector, which contains tyrosine-to-phenylalanine Tyr-Phe mutations at positions Y733F, Y500F, and Y730F, respectively, is used. In some embodiments, a triple-mutant AAV8 vector, which contains tyrosine-to-phenylalanine Tyr-Phe mutations at positions Y447F, Y733F, and T494V (e.g., AAV8 (Y447F+Y733F+T494F)) is used.

In some embodiments, the rAAV particles of the disclosure comprises a transgene, or heterologous nucleic acid, that is too large for delivery in standard AAV systems. In some embodiments, the transgene is hABCA4, which encodes a human ABCA4 polypeptide. In some embodiments, an hABCA4 polypeptide comprises the amino acid sequence shown in SEQ ID NO: 2 or a functional fragment or a variant thereof. In some embodiments, the hABCA4 polypeptide is encoded by the nucleotide sequence set forth in SEQ ID NO: 1.

In some embodiments, administration of any of the disclosed polynucleotide vectors to the eye of a subject in need thereof restores vision loss, partially or completely. The transgene may comprise a human ABCA4. In some embodiments, these administrations may reduce lipofuscin buildup in the macula of a subject.

In some embodiments, the production of the therapeutic agent encoded by the transgene of any of the disclosed polynucleotide vector systems in cells of the eye (such as retinal cells or RPE cells) provides one or more of the following therapeutic endpoints: a) preserves one or more photoreceptor cells or one or more RPE cells, b) restores one or more rod- and/or cone-mediated functions, c) restores visual behavior in one or both eyes, or d) any combination thereof. In particular embodiments, production of the therapeutic agent in the disclosed methods preserves one or more PR cells, such as retinal ganglion cells, bipolar cells, Müller glial cells or astrocyte cells, or RPE cells.

In some embodiments, production of the therapeutic agent persists in the one or more photoreceptor cells or the one or more RPE cells substantially for a period of at least three months, at least six months, at least nine months, or at least a year or more, following an initial administration of any of the disclosed rAAV polynucleotide vector system into the one or both eyes of the mammal.

In this manner, the polynucleotide vector systems and compositions thereof of the disclosure may be used to treat or ameliorate symptoms of Stargardt Disease in the eyes of the subject. In some embodiments, symptoms of Stargardt Disease include problems with night vision, problems with color vision, or abnormal accumulation of lipofuscin in the macula of a subject.

In some embodiments, the disclosure provides rAAV nucleic acid vectors that include at least a first nucleic acid segment that encodes one or more diagnostic or therapeutic agents that alter, inhibit, reduce, prevent, eliminate, or impair the activity of one or more endogenous biological processes in a mammalian cell suitably transformed with the vector of interest. In certain embodiments, such diagnostic or therapeutic agents may include a molecule that selectively inhibits or reduces the effects of one or more metabolic processes, dysfunctions, disorders, or diseases. In certain embodiments, the defect may be caused by injury or trauma to the mammal for which treatment is desired. In other embodiments, the defect may be caused the over-expression of an endogenous biological compound, while in other embodiments still; the defect may be caused by the under-expression or even lack of one or more endogenous biological compounds.

Regulatory Elements of rAAV Vectors

Any of the vector systems of the disclosure may include regulatory elements that are functional in the intended host cell in which the vector is to be expressed. Regulatory elements include, for example, promoters, transcription termination sequences, translation termination sequences, enhancers, and polyadenylation elements.

Any of the vector systems of the disclosure may include a promoter sequence operably linked to a nucleotide sequence encoding a desired polypeptide. Promoters contemplated for use in the disclosure include, but are not limited to, cytomegalovirus (CMV) promoter, SV40 promoter, human ABCA4 gene-derived promoter, Rous sarcoma virus (RSV) promoter, chimeric CMV/chicken β-actin promoter (CBA) and the truncated form of CBA (smCBA). In some embodiments, the promoter comprises a smCBA promoter (SEQ ID NO: 23), or a sequence at least 85%, 86%, 87%, 88%, 89%, 90%, 91%, 92%, 93%, 94%, 95%, 96%, 97%, 98% or 99% identical to SEQ ID NO: 23. Additional photoreceptor-specific, human rhodopsin kinase (hGRK1) promoter, rod specific IRBP promoter, VMD2 (vitelliform macular dystrophy/Best disease) promoter, a RPE-specific vitelliform macular dystrophy-2 [VMD2] promoter, and EF1-alpha promoter sequences are also contemplated to be useful in the practice of various aspects of the disclosure. Non-limiting examples of photoreceptor-cell-specific promoters include, but are not limited to, hGRK1, IRBP, rod opsin, NRL, GNAT2e-IRBP, L/M opsin, and cone arrestin promoters.

In particular embodiments, the promoter is a chimeric CMV-β-actin promoter. In particular embodiments, the promoter is a tissue-specific promoter that shows selective activity in one or a group of tissues but is less active or not active in other tissue. In particular embodiments, the promoter is a photoreceptor-specific promoter. In a further embodiment, the promoter is preferably a cone cell-specific promoter or a rod cell-specific promoter, or any combination thereof. In particular embodiments, the promoter is the promoter for a human ABCA4 gene. In a further embodiment, the promoter comprises a cone transducin a (TαC) gene-derived promoter. In particular embodiments, the promoter is a human GNAT2-derived promoter. Other promoters contemplated within the scope of the disclosure include, without limitation, a rhodopsin promoter (human or mouse), a cGMP-phosphodiesterase β-subunit promoter, a retinitis pigmentosa-specific promoter, an RPE cell-specific promoter [such as a vitelliform macular dystrophy-2 (VMD2) promoter (Best1)], or any combination thereof.

Promoters can be incorporated into a vector using standard techniques known to those of ordinary skill in the molecular biology and/or virology arts. Multiple copies of promoters, and/or multiple distinct promoters can be used in the vectors of the disclosure. In one such embodiment, a promoter may be positioned about the same distance from the transcription start site as it is from the transcription start site in its natural genetic environment, although some variation in this distance is permitted, of course, without a substantial decrease in promoter activity. In the practice of the disclosure, one or more transcription start site(s) are typically included within the disclosed vectors.

The vectors of the disclosure may further include one or more transcription termination sequences, one or more translation termination sequences, one or more signal peptide sequences, one or more internal ribosome entry sites (IRES), and/or one or more enhancer elements, or any combination thereof. Transcription termination regions can typically be obtained from the 3′-untranslated region of a eukaryotic or viral gene sequence. Transcription termination sequences can be positioned downstream of a coding sequence to provide for efficient termination.

Any of the disclosed polynucleotide vectors may also further include one or more post-transcriptional regulatory sequences or one or more polyadenylation signals, including, for example, but not limited to, a woodchuck hepatitis virus post-transcription regulatory element (WRPE), a polyadenylation signal sequence, or an intron/exon junctions/splicing signals, or any combination thereof.

Signal peptide sequences are amino-terminal peptidic sequences that encode information responsible for the location of an operably-linked polypeptide to one or more post-translational cellular destinations, including, for example, specific organelle compartments, or to the sites of protein synthesis and/or activity, and even to the extracellular environment.

Enhancers-cis-acting regulatory elements that increase gene transcription—may also be included in one of the disclosed AAV-based vector systems. A variety of enhancer elements are known to those of ordinary skill in the relevant arts, and include, without limitation, a CaMV 35S enhancer element, a cytomegalovirus (CMV) early promoter enhancer element, an SV40 enhancer element, as well as combinations and/or derivatives thereof. One or more nucleic acid sequences that direct or regulate polyadenylation of the mRNA encoded by a structural gene of interest, may also be optionally included in one or more of the vectors of the disclosure.

### Host Cells and Methods for Transducing Cells

The disclosure provides host cells comprising vectors of the disclosed polynucleotide vector systems. In some embodiments, an isolated host cell comprising a dual polynucleotide vector system is provided.

Examples of suitable host cells that comprise any of the disclosed dual vector systems include, but are not limited to, photoreceptor cells, cone cells, rod cells, retinal cells (e.g., ganglion cells, retinal pigment epithelium cells), or any combination thereof. Examples of retinal cells include retinal ganglion cells (RGCs), Muller cells, astrocytes, and bipolar cells.

The disclosure also provides methods for expressing or transducing a selected polypeptide in a cell. In particular embodiments, the method comprises incorporating in the cell an AAV-based, dual vector system as disclosed herein, wherein the vector system includes a polynucleotide sequence that encodes a selected polypeptide and of interest, and expressing the polynucleotide sequences in the cell.

In some embodiments, the selected polypeptide may be a polypeptide that is heterologous to the cell. In some embodiments, the cell is a mammalian cell, and preferably, a human cell. In some embodiments, the cell is a human photoreceptor cell, and preferably a human photoreceptor cone cell or a photoreceptor rod cell. In particular embodiments, the cell expresses a wild type, functional, and/or biologically-active ABCA4 polypeptide that is encoded by a nucleic acid segment present in a vector system as disclosed herein. In some embodiments, the ABCA4 polypeptide is encoded by the nucleotide sequence shown in SEQ ID NO: 1.

Accordingly, in certain embodiments, the disclosure provides for methods for transducing or expressing a polynucleotide vector system in one or more photoreceptor cells or one or more RPE cells of a mammal (e.g., a human). In an overall and general sense, such a method includes administering (for example, directly administering subretinally) to one or both eyes of the mammal one or more of the rAAV particles disclosed herein, wherein the polynucleotide further comprises at least a first polynucleotide that comprises a PR- or an RPE-cell-specific promoter operably linked to at least a first heterologous nucleic acid segment that encodes a therapeutic agent, for a time effective to produce the therapeutic agent in the one or more PR cells or RPE cells of the mammal. In certain embodiments, the therapeutic agent is stably expressed in a photoreceptor cell, retinal pigment epithelium cell, retinal ganglion cell, bipolar cell, Müller glial cell or astrocyte cell, or combinations thereof.

### Methods of Treatment and Transduction

In some aspects, the disclosure provides methods for treating or ameliorating a disease or condition, such as an eye disease, in a human or animal using gene therapy and an AAV-based dual vector system of the disclosure. In particular embodiments, a method of the disclosure comprises administering a vector system of the disclosure that encodes a polypeptide that provides for treatment or amelioration of the disease or condition. In particular embodiments, the vectors of the disclosure are provided in an AAV virus or virion. The vector system can be administered in vivo or ex vivo.

In some embodiments, a vector system of the disclosure is administered in a recombinant AAV particle by parenteral administration, such as intravitreal, subretinal, intravenous, intramuscular, intraocular, or intranasal injection. In particular embodiments, a vector system of the disclosure is administered to the human or animal by intraocular, intravitreal or subretinal injection.

In some embodiments, the disease, disorder or condition to be treated is Stargardt Disease.

The disclosed dual vector systems may be introduced into one or more selected mammalian cells using any one or more of the methods. Such methods include, without limitation, transfection, microinjection, electroporation, lipofection, cell fusion, and calcium phosphate precipitation, as well as biolistic methods. In particular embodiments, the vectors of the disclosure may be introduced in vivo, including, for example, by lipofection (for example, DNA transfection via liposomes prepared from one or more cationic lipids). Synthetic cationic lipids (LIPOFECTIN®, Invitrogen Corp., La Jolla, CA, USA) may be used to prepare liposomes that will encapsulate the vectors to facilitate their introduction into one or more selected cells. A vector system of the disclosure can also be introduced in vivo as “naked” DNA.

In an overall and general sense, the disclosed methods include at least the step of administering to one or both eyes of the mammal in need thereof, one or more of the disclosed rAAV particles herein, in an amount and for a time sufficient to treat or ameliorate the one or more symptoms of the disease, the disorder, the dysfunction, the injury, the abnormal condition, or the trauma in the mammal. In some embodiments, the mammal is a human. In some embodiments, the human is a neonate, a newborn, an infant, or a juvenile. In the practice of the present disclosure, it is contemplated that suitable patients will include, for example, humans that have, are suspected of having, are at risk for developing, or have been diagnosed with one or more retinal disorders, diseases, or dystrophies, including, without limitation, retinal disorders, diseases, and dystrophies that are genetically linked, or inheritable.

In some aspects, the present disclosure provides methods of use of the particles, vectors, virions, expression systems, compositions, and host cells described herein in a method for treating or ameliorating the symptoms, or in the preparation of medicaments for, treating or ameliorating the symptoms of various deficiencies in an eye of a mammal, and in particular one or more deficiencies in human photoreceptors or RPE cells. In some embodiments, the subject in need thereof suffers from Stargardt Disease. In some embodiments, the subject suffers from disease or condition of the eye that can benefit from treatment with gene therapy comprising an ABCA4 polynucleotide.

In some embodiments, administration of any of the disclosed vectors, virions, or compositions to a subject in need thereof provides a partial or complete restoration of melanosome migration in retinal pigment epithelium (RPE) cells. In some embodiments, administration of any of the polynucleotide vector systems, virions, or compositions provides a partial or complete restoration of vision loss.

Such methods generally may involve intravitreal or subretinal administration to one or both eyes of a subject in need thereof, one or more of the disclosed particles vectors, virions, host cells, or compositions, in an amount and for a time sufficient to treat or ameliorate the symptoms of such a deficiency in the affected mammal. The methods may also encompass prophylactic treatment of animals suspected of having such conditions, or administration of such compositions to those animals at risk for developing such conditions either following diagnosis, or prior to the onset of symptoms.

### Pharmaceutical Compositions and Kits

The disclosure also provides pharmaceutical compositions comprising a vector system of the disclosure in combination with a pharmaceutically acceptable carrier. The dose administered to a patient, particularly a human, in the context of the disclosure should be sufficient to achieve a therapeutic response in the patient over a reasonable timeframe, without lethal toxicity, and preferably causing no more than an acceptable level of side effects or morbidity. In some embodiments, dosage will depend upon a variety of factors including the condition (health) of the subject, the body weight of the subject, kind of concurrent treatment, if any, frequency of treatment, therapeutic ratio, as well as the severity and stage of the pathological condition.

The disclosure also provides kits comprising a vector system of the disclosure in one or more containers. Kits of the disclosure can optionally include pharmaceutically acceptable carriers and/or diluents. In particular embodiments, a kit of the disclosure includes one or more other components, adjuncts, or adjuvants as described herein. In particular embodiments, a kit of the disclosure includes instructions or packaging materials that describe how to administer a vector system contained within the kit to a selected mammalian recipient.

Containers of the disclosed kits may be of any suitable material, e.g., glass, plastic, metal, etc., and of any suitable size, shape, or configuration. In particular embodiments, a vector system of the disclosure is provided in the kit as a solid. In another embodiment, a vector system of the disclosure is provided in the kit as a liquid or solution. In some embodiments, the kits may include one or more ampoules or syringes that contain a vector system of the disclosure in a suitable liquid or solution form.

The disclosure also provides for the use of the buffers and compositions disclosed herein in the manufacture of a medicament for treating, preventing or ameliorating the symptoms of a disease, disorder, dysfunction, injury or trauma, including, but not limited to, the treatment, prevention, and/or prophylaxis of a disease, disorder or dysfunction, and/or the amelioration of one or more symptoms of such a disease, disorder or dysfunction.

To express a therapeutic agent in accordance with the present disclosure one may prepare a rAAV particle that comprises a therapeutic agent-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (for example, 3′ of) the chosen promoter. The “upstream” promoter stimulates transcription of the DNA and promotes expression of the encoded polypeptide. This is the meaning of “recombinant expression” in this context. In some embodiments, recombinant vector constructs are those that include a capsid-protein modified rAAV vector that contains an RPE cell- or a photoreceptor cell-specific promoter, operably linked to at least one nucleic acid segment encoding one or more diagnostic, and/or therapeutic agents.

When the use of such vectors is contemplated for introduction of one or more exogenous proteins, polypeptides, peptides, ribozymes, and/or antisense oligonucleotides, to a particular cell transfected with the vector, one may employ the rAAV particles disclosed herein to deliver one or more exogenous polynucleotides to a selected host cell, e.g., to one or more selected cells within the mammalian eye.

In some embodiments, the disclosure provides formulations of one or more viral-based compositions disclosed herein in pharmaceutically acceptable solutions for administration to a cell or an animal, either alone or in combination with one or more other modalities of therapy, and in particular, for therapy of human cells, tissues, and diseases affecting man.

In some embodiments, rAAV particles described herein may be administered in combination with other agents as well, such as, e.g., proteins or polypeptides or various pharmaceutically-active agents, including one or more systemic or topical administrations of therapeutic polypeptides, biologically active fragments, or variants thereof.

Formulation of pharmaceutically-acceptable buffer, excipients and carrier solutions is well known to those of skill in the art, as is the development of suitable dosing and treatment regimens for using the particular compositions described herein in a variety of treatment regimens, including e.g., oral, parenteral, intraocular (e.g., subretinal or intravitreal), intravenous, intranasal, intra-articular, intracochlear and intramuscular administration and formulation.

The term “excipient” refers to a diluent, adjuvant, carrier, or vehicle with which the rAAV particle is administered. Such pharmaceutical excipients can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Non-limiting examples of excipients and vehicles include, but are not limited to, HA, BSS, artificial CSF, PBS, Ringer's lactate solution, TMN200 solution, polysorbate 20, and poloxamer 100.

Non-limiting examples of compositions may include rAAV particles or nucleic acid vectors either alone, or in combination with one or more additional active ingredients, which may be obtained from natural or recombinant sources or chemically synthesized.

Methods of Manufacturing rAAV Particles

Recombinant adeno-associated virus (rAAV) vectors have been used successfully for in vivo gene transfer in numerous pre-clinical animal models of human disease, and have been used successfully for long-term expression of a wide variety of therapeutic genes. AAV vectors have also generated long-term clinical benefit in humans when targeted to immune-privileged sites, for example, ocular delivery for Leber congenital amaurosis. A major advantage of this vector is its comparatively low immune profile, eliciting only limited inflammatory responses and, in some cases, even directing immune tolerance to transgene products. Nonetheless, the therapeutic efficiency, when targeted to non-immune privileged organs, has been limited in humans due to antibody and CD8+ T cell responses against the viral capsid, while in animal models, adaptive responses to the transgene product have also been reported. These results suggested that immune responses remain a concern for AAV vector-mediated gene transfer.

Adeno-associated virus (AAV) is considered the optimal vector for ocular gene therapy due to its efficiency, persistence and low immunogenicity. Identifying vectors capable of transducing PRs via the vitreous has historically relied on identifying which serotypes have native tropism for this cell type following local delivery. Several serotypes have been used to successfully target transgene to PRs following subretinal injection (including, e.g., AAV2, AAV5 and AAV8) with all three demonstrating efficacy in experiments performed across multiple mammalian species (e.g., mouse, rat, dog, pig and non-human primate).

Studies comparing their relative efficiency following subretinal delivery in the rodent show that both AAV5 and AAV8 transduce PRs more efficiently than AAV2, with AAV8 being the most efficient. It was previously shown that AAV2 and AAV8 vectors containing point mutations of surface-exposed tyrosine residues (tyrosine to phenylalanine, Y-F) display increased transgene expression in a variety of retinal cell types relative to unmodified vectors following both subretinal and intravitreal injection. Of the vectors initially tested by those authors, an AAV2 triple mutant (designated “triple Y-F”) exhibited the highest transduction efficiency following intravitreal injection, whereas an AAV2 quadruple mutant (“quad Y-F”) exhibited the property of enhanced transduction of outer retina.

Further improvements in transduction efficiency have been achieved via directed mutagenesis of surface exposed threonine (T) or serine(S) residues to non-native amino acids at one of more of those amino acids. Both Y-F and T-V/T-A mutations have been shown to increase efficiency by decreasing phosphorylation of capsid and subsequent ubiquitination as part of the proteosomal degradation pathway. It has been found that the transduction profile of intravitreally-delivered AAV is heavily dependent upon the injection procedure itself. Due to the small size of the mouse eye, it is not uncommon for trans-scleral, intravitreal injections to result in damage to the retina that might allow delivery of some vector directly to the subretinal space.

In some embodiments, rAAV nucleic acid vectors useful according to the disclosure include single-stranded (ss) or self-complementary (sc) AAV nucleic acid vectors, such as single-stranded or self-complementary recombinant viral genomes.

Methods of producing rAAV particles and nucleic acid vectors and commercially available (see, e.g., plasmids and kits available from ATCC and Cell Biolabs, Inc.). For example, a plasmid containing the nucleic acid vector sequence may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids includes a first helper plasmid comprising a rep gene and a cap gene and a second helper plasmid comprising a Ela gene, a Elb gene, a E4 gene, a E2a gene, and a VA gene. In some embodiments, the rep gene is a rep gene derived from AAV2 and the cap gene is derived from AAV2 and includes modifications to the gene in order to produce a modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG (R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, CA; and Addgene, Cambridge, MA; pxx6.

A non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

### Definitions

In accordance with the disclosure, polynucleotides, nucleic acid segments, nucleic acid sequences, and the like, include, but are not limited to, DNAs (including, but not limited to, genomic and/or extragenomic DNAs), genes, peptide nucleic acids (PNAs), RNAs (including, but not limited to, rRNAs, mRNAs, and/or tRNAs), nucleosides, as well as one or more nucleic acid segments obtained from natural sources, chemically synthesized, genetically modified, or otherwise prepared or synthesized in whole or in part by the hand of man.

Unless defined otherwise, all technical and scientific terms used herein have the same meaning as commonly understood by one of ordinary skill in the art. Although any methods and compositions similar or equivalent to those described herein can be used in the practice or testing of the disclosure, the preferred methods and compositions are described herein.

In some embodiments, the terms “nucleic acid” and “polynucleotide sequence” refer to a deoxyribonucleotide or ribonucleotide polymer in either single- or double-stranded form, and unless otherwise limited, encompass known analogs of natural nucleotides that can function in a similar manner as naturally occurring nucleotides. Polynucleotide sequences may include both full-length sequences, as well as shorter sequences derived from the full-length sequences. A particular polynucleotide sequence may include the degenerate codons of the native sequence or sequences that may be introduced to provide codon preference in a specific host cell. Polynucleotide sequences may include sequences that specifically hybridize with the sequences coding for a peptide of the disclosure. Polynucleotide may include both the sense and antisense strands, either as individual strands or in the duplex.

Fragments and variants of a polynucleotide of the disclosure can be generated as described herein and tested for the presence of function. Fragments and variants of a polynucleotide or polypeptide of the disclosure can be tested to determine whether the fragment or variant retains functional activity that is the same or similar to a full-length or a non-variant polynucleotide or polypeptide, such as an ABCA4 polynucleotide or polypeptide.

Also within the scope of the disclosure are polynucleotides that have the same, or substantially the same, nucleotide sequence of a polynucleotide described herein, except for the presence of one or more nucleotide substitutions, additions, or deletions within the sequence of the polynucleotide, so long as these variant polynucleotides retain substantially the same relevant functional activity as the polynucleotides herein (for example, they encode a protein having the same amino acid sequence or the same functional activity as one of the polynucleotides specifically described herein). Thus, the polynucleotides disclosed herein should also be understood to include variants and fragments thereof.

There can be a number of variant sequences of a gene or polynucleotide found in nature, in addition to those variants that may be artificially prepared or synthesized by an ordinary-skilled artisan in a laboratory environment. The polynucleotides of the disclosure encompasses those specifically described herein, as well as any natural variants thereof, as well as any variants which can be created artificially, so long as those variants retain the desired biological activity.

Also within the scope of the disclosure are polynucleotides which have the same nucleotide sequences of a polynucleotide described herein except for nucleotide substitutions, additions, or deletions within the sequence of the polynucleotide, as long as these variant polynucleotides retain substantially the same relevant biological activity as the polynucleotides specifically described herein. Thus, the polynucleotides disclosed herein should be understood to include variants and fragments, as discussed above, of the specifically described sequences.

Polynucleotides described herein can also be defined in terms of more particular identity and/or similarity ranges with those described herein. The sequence identity may be greater than 60%, greater than 75%, greater than 80%, greater than 90%, or can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% or greater as compared to a sequence described herein.

Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, word-length=12, to obtain sequences with the desired percent sequence identity. To obtain gapped alignments for comparison purposes, Gapped BLAST can be used. When utilizing BLAST and Gapped BLAST programs, the default parameters of the respective programs (NBLAST and XBLAST) can be used in accordance with published methods.

The disclosure also contemplates those polynucleotide molecules having sequences that are sufficiently homologous with the polynucleotide sequences of the disclosure to permit hybridization with that sequence under standard stringent conditions and standard methods. As used herein, “stringent” conditions for hybridization refers to conditions wherein hybridization is typically carried out overnight at 20-25 degrees Celsius below the melting temperature (Tm) of the DNA hybrid in 6×SSPE, 5×Denhardt's solution, and 0.1% SDS, containing 0.1 mg/mL of a suitable non-specific denatured DNA.

In some embodiments, an effective amount refers to an amount that is capable of treating or ameliorating a disease or condition or otherwise capable of producing an intended therapeutic effect.

In some embodiments, operably linked means that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

In some embodiments, a promoter is a region or regions of a nucleic acid sequence that regulates transcription. Non-limiting examples of promoters provided herein include, but are not limited to, a CMV promoter, an EF-1 alpha promoter, a cone arrestin promoter, a chimeric CMV β actin promoter (CBA), a truncated chimeric CMV β actin (smCBA) promoter, a smCBA promoter, a human ABCA4 gene-derived promoter, a TαC gene-derived promoter, a rhodopsin promoter, a cGMP-phosphodiesterase β-subunit promoter, human or mouse rhodopsin promoter, a hGRK1 promoter, a rod specific IRBP promoter, and a VMD2 promoter.

In some embodiments, a regulatory element refers to a region or regions of a nucleic acid sequence that regulates transcription. Non-limiting examples of regulatory elements include, but are not limited to, enhancers, post-transcriptional elements, transcriptional control sequences, and such like.

In some embodiments, substantially denotes a characteristic of a nucleic acid or an amino acid sequence, wherein a selected nucleic acid or amino acid sequence has at least about 70 or about 75 percent sequence identity as compared to a selected reference nucleic acid or amino acid sequence. In some cases, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. Highly homologous sequences often share greater than at least about 96, 97, 98, or 99 percent sequence identity between the selected sequence and the reference sequence to which it was compared.

The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. In the case of sequence homology of two or more polynucleotide sequences, the reference sequence may comprise at least about 18-25 nucleotides, at least about 26 to 35 nucleotides, or at least about 40, 50, 60, 70, 80, 90, or even 100 or so nucleotides.

When highly-homologous fragments are desired, the extent of percent identity between the two sequences may be at least about 80%, at least about 85%, or about 90% or 95% or higher, as readily determined by a sequence comparison algorithm, such as e.g., the FASTA program analysis.

In some embodiments, subject describes an organism, including mammals such as primates, to which treatment with the compositions according to the disclosure can be provided. Mammalian species that can benefit from the disclosed methods of treatment include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

In some embodiments, treatment or any grammatical variation thereof (e.g., treat, treating, and treatment, etc.), includes but is not limited to, alleviating a symptom of a disease or condition; and/or reducing, suppressing, inhibiting, lessening, ameliorating or affecting the progression, severity, and/or scope of a disease or condition.

In some embodiments, vector refers to a nucleic acid molecule (typically one containing DNA) that is capable of replication in a suitable host cell, or one to which another nucleic acid segment can be operatively linked so as to facilitate replication of the operably-linked nucleic acid segment. Exemplary vectors include, without limitation, plasmids, cosmids, viruses and the like.

In some embodiments, variant refers to a molecule (e.g., a capsid polynucleotide) having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type capsid polynucleotide. Variants of a protein molecule, e.g., a capsid, may contain modifications to the amino acid sequence (e.g., having 1, 2, 3, 4, 5, 6, 7, 8, 9, 10, 10-15, or 15-20 amino acid substitutions) relative to the wild type protein sequence, which arise from point mutations installed into the nucleic acid sequence encoding the capsid protein. These modifications include chemical modifications as well as truncations.

Variants of a nucleic acid molecule, e.g., a polynucleotide vector system, may contain modifications to the sequence (e.g., having 1, 2, 3, 4, 5, 6, 7, 8, 9, 10, 10-15, or 15-20 nucleotide substitutions) relative to the wild type nucleic acid sequence. These modifications may comprise truncations at a 5′ terminus of a 3′ terminus.

### Examples

The following examples are included to demonstrate example embodiments of the disclosure.

EXAMPLE 1 describes a non-limiting dual-AAV vector system, which utilizes alkaline phosphatase (AP) splicing. In each system shown in FIG. 1, there are two vectors, each comprising an AP homology domain (APHead). In one of the systems, labeled “Dual AAV-ABCA4 SP2 (ex20/21)”, Vector A contains the coding sequence corresponding to the amino-terminal portion of the hABCA4 cDNA through exon 20 (SEQ ID NO: 13) and the splice-donor site (SEQ ID NO: 24), followed by the APHead intron (SEQ ID NO: 22). Vector B contains the APHead intron (SEQ ID NO: 22), followed by the splice-acceptor site (SEQ ID NO: 25), then the carboxyl-terminal portion of the hABCA4 cDNA from the beginning of exon 21 (SEQ ID NO: 15). Upon co-delivery to suitable mammalian host cells, the DNA of vectors A and B recombine to form a reconstituted full-length gene cassette. The resulting RNA transcript will then ‘splice out’ the intron (SEQ ID NO: 22). Alternatively, recombination and formation of the gene cassette can occur via the AAV ITRs. In this case, the RNA transcript will ‘splice out’ the intron-ITR-intron motif (SEQ ID NO: 29). In both cases, however, the resulting mRNA (SEQ ID NO: 1) is that of full-length hABCA4, which is then translated into hABCA4 protein (SEQ ID NO: 2). Similarly, the system labeled “Dual AAV-ABCA4 SP1 (ex19/20)” comprises two vectors—a first vector containing the coding sequence corresponding to the amino-terminal portion of the hABCA4 cDNA through exon 19 (SEQ ID NO: 9), and a second vector containing the coding sequence corresponding to the carboxyl-terminal portion of the hABCA4 cDNA from the beginning of exon 20 (SEQ ID NO: 11). Also shown in FIG. 1 is the system labeled “Dual AAV-ABCA4 SP3 (ex21/22)”, which comprises two vectors—a first vector containing the coding sequence corresponding to the amino-terminal portion of the hABCA4 cDNA through exon 21 (SEQ ID NO: 17), and a second vector containing the coding sequence corresponding to the carboxyl-terminal portion of the hABCA4 cDNA from the beginning of exon 22 (SEQ ID NO: 19).

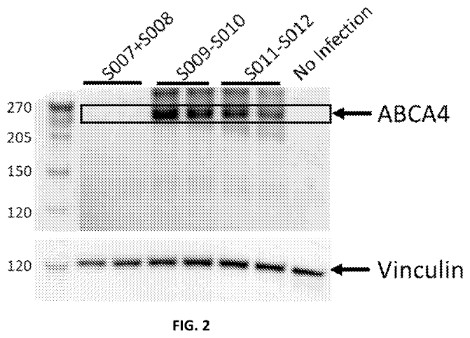

EXAMPLE 2 describes in vitro performance of three non-limiting dual vector systems. Each of the three dual AAV-ABCA4 vectors were packaged into AAV44.9 (E531D) (SEQ ID NO: 28) by triple transfection of HEK293 cells: S007+S008 (SP1, SEQ ID NOS: 3 and 4, respectively); S009+S010 (SP2, SEQ ID NOS: 5 and 6, respectively); and S011+S012 (SP3, SEQ ID NOS: 7 and 8, respectively). Resulting vector pairs were used to infect AAVR cells at an MOI of 100,000. At 3 days post infection, cells were harvested and total protein was extracted. Protein samples were run on immunoblot and probed with antibody to ABCA4 (FIG. 2). Both SP2 (S009-S010) and SP3 (S011-S012) dual AAV-ABCA4 vector pairs lead to high levels of full length ABCA4 expression in vitro.

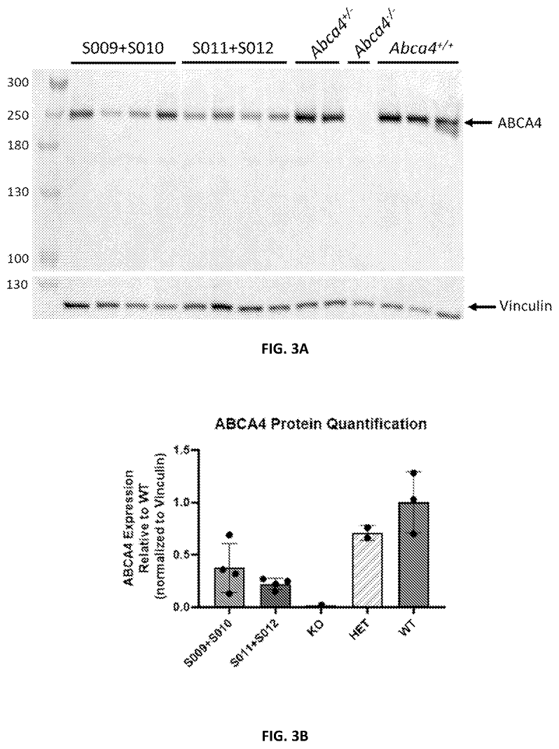

EXAMPLE 3 compares ABCA4 expression in Abca4−/− knockout mice resulting from bilateral subretinal injection of the three dual AAV-ABCA4 vector expression cassettes as described in Examples 1 and 2 and shown in FIG. 1. Levels of dual AAV vector-mediated ABCA4 were compared to endogenous levels in heterozygous and wild-type mice. Results are shown in FIGS. 3A and 3B. No ABCA4 expression was observed in retinas from mice injected with S007+S010 samples (SEQ ID NOS: 3 & 4; data not shown). S009/S010 (SEQ ID NOS: 5 & 6) produced highest levels of ABCA4 expression. S009/S010 produced a range of 13-69% of wild type levels of ABCA4, with an average of 37% of wild type levels of ABCA4. The S011/S012 vector pair (SEQ ID NOS: 7 & 8) produced a range of 15-27% of wild type levels of ABCA4, with an average of 22% of wild type levels of ABCA4. These results confirm that the dual AAV vectors having the split point between exons 20 and 21 produce robust levels of full length ABCA4 in retinas of Abca4−/− mice.

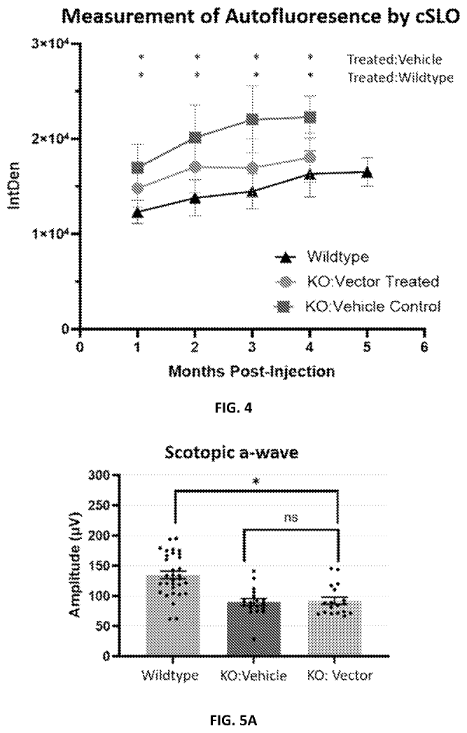

EXAMPLE 4 shows in vivo data from Abca4−/− mice that were subretinally injected with AAV44.9 (E531D) (capsid encoded by SEQ ID NO: 28) particles comprising S009-S010 vector pair (SEQ ID NOS: 5 & 6). Treatment conferred a reduction of autofluorescence in Abca4−/− knockout mouse retinas. In both Stargardt's disease patients and Abca4−/− knockout mouse retinas, toxic autofluorescent bisretinoids accumulate and can be measured by scanning laser ophthalmoscopy (cSLO). Subretinal injections were performed in one eye of Abca4−/− knockout mice. The contralateral eye was injected with vehicle alone. Uninjected wild type mice were also included as a comparison. In FIG. 4, measurements of average pixel intensities from cSLO images of dual vector vs. vehicle-injected Abca4−/− mice, or uninjected WT controls are graphed at multiple timepoints. A reduction in autofluorescence was observed in vector injected Abca4−/− eyes compared to vehicle injected eyes at all time points post-injection. These results demonstrate that ABCA4 generated from the S009-S010 vector pair functions to reduce the retinal autofluorescence phenotype of Abca4−/− mice.

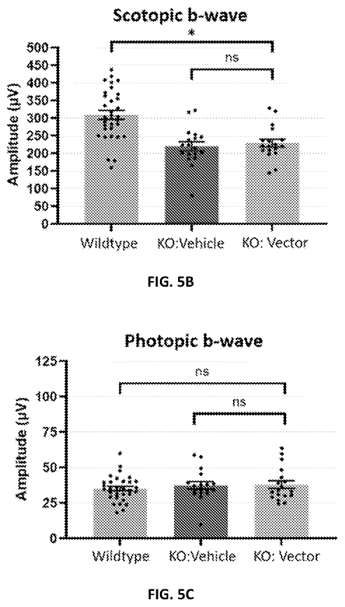

EXAMPLE 5 shows electroretinogram (ERG) data from Abca4−/− mice subretinally injected with AAV44.9 (E531D) (capsid encoded by SEQ ID NO: 28) particles comprising S009-S010 dual vector pair (SEQ ID NOS: 5 & 6). ERG recordings were performed 2-months post injection under scotopic (rod-mediated) and photopic (cone-mediated) conditions and maximum a-wave (photoreceptor) (FIG. 5A) and b-wave (bipolar cell) (FIG. 5B-C) amplitudes were calculated/averaged. ERG recordings were also performed on uninjected wildtype mouse eyes. No significant differences were observed in ERG amplitudes between the S009-S010 vector injected vs. vehicle injected eyes of Abca4−/− mice. These data demonstrate that, at 2-months post injection, the S009-S010 dual AAV vectors are well tolerated and do not result in any reduction in retinal function.

### EQUIVALENTS

It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g, “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

### Embodiments

Also disclosed herein are the following embodiments:
1. A polynucleotide vector system for providing ABCA4 or functional portions thereof, comprising
   i) a first AAV vector polynucleotide comprising an inverted terminal repeat at each end of the polynucleotide, and between the inverted terminal repeats a promoter followed by a partial ABCA4 coding sequence that encodes an N-terminal part of ABCA4 followed by a splice donor site and a homology region, and
   ii) a second AAV vector polynucleotide comprising an inverted terminal repeat at each end of the polynucleotide, and between the inverted terminal repeats a homology region and a splice acceptor site and a partial ABCA4 coding sequence that encodes a C-terminal part of ABCA4,
wherein the homology region in the first AAV vector and the homology region in the second AAV vector comprise a polynucleotide sequence that overlaps, and
wherein a split point between the first AAV vector polynucleotide and the second AAV vector polynucleotide sequences is between exon 20 and exon 21 of the ABCA4 gene.
2. The polynucleotide vector system of embodiment 1, wherein the homology region in the first AAV vector and the homology region in the second AAV vector is an alkaline phosphatase homology region.
3. The polynucleotide vector system of embodiment 1 or 2, wherein the N-terminal part of ABCA4 comprises at least 85% sequence identity with SEQ ID NO. 14.
4. The polynucleotide vector system of embodiment 1 or 2, wherein the C-terminal part of ABCA4 comprises at least 85% sequence identity with SEQ ID NO. 16.
5. The polynucleotide vector system of any one of embodiments 1-4, wherein the first AAV vector polynucleotide sequence comprises a nucleotide sequence with at least 85% sequence identity with SEQ ID NO. 13.
6. The polynucleotide vector system of any one of embodiments 1-5, wherein the first AAV vector polynucleotide sequence comprises a nucleotide sequence with at least 85% sequence identity with SEQ ID NO. 15.
7. The polynucleotide vector system of any one of embodiments 1-6, wherein the first AAV vector polynucleotide comprises at least 85% sequence identity with the nucleotide sequence of SEQ ID NO: 5.
8. The polynucleotide vector system of any one of embodiments 1-7, wherein the second AAV vector polynucleotide comprises at least 85% sequence identity with the nucleotide sequence of SEQ ID NO: 6.
9. The polynucleotide vector system of any one of embodiments 1-8, wherein the first AAV vector polynucleotide comprises the nucleotide sequence of SEQ ID NO: 5.
10. The polynucleotide vector system of any one of embodiments 1-9, wherein the second AAV vector polynucleotide comprises the nucleotide sequence of SEQ ID NO: 6.
11. The polynucleotide vector system of any one of embodiments 1-10, wherein the first AAV vector polynucleotide comprises the nucleotide sequence of SEQ ID NO: 5, and the second AAV vector polynucleotide comprises the nucleotide sequence of SEQ ID NO: 6.
12. The polynucleotide vector system of any one of embodiments 1-11, wherein the polynucleotide sequence that is shared between vectors is about 50 to about 500 nucleotides, or about 200 to 300 nucleotides in length.
13. The polynucleotide vector system of any one of embodiments 1, 3-6, or 12, wherein the homology region comprises an intron sequence.
14. The polynucleotide vector system of embodiment 13, wherein the intron sequence comprises a sequence of an intron naturally present in the genomic sequence of the gene encoding an ABCA4 polypeptide.
15. The polynucleotide vector system of embodiment 13 or 14, wherein the intron sequence is the AK sequence of the F1 phage.
16. The polynucleotide vector system of any one of embodiments 13-16, wherein the intron sequence comprises a synthetic alkaline phosphatase (AP) intron or an intron derived from MYO7A.
17. The polynucleotide vector system of any one of embodiments 1-16, further comprising one or more nucleotide substitutions in one or more noncoding regions of the first AAV vector polynucleotide and/or the second AAV vector polynucleotide.
18. The polynucleotide vector system of any embodiment 17, further comprising one or more nucleotide substitutions in one or more noncoding regions of the first AAV vector polynucleotide.
19. The polynucleotide vector system of embodiment 18, wherein the one or more noncoding sequences comprise the alkaline phosphatase (AP) head sequence.
20. The polynucleotide vector system of embodiment 17 or embodiment 18, wherein the one or more noncoding sequences comprise the AP intron.
21. The polynucleotide vector system of embodiment 18, wherein the one or more noncoding sequences comprise a 3′ untranslated region (UTR) between the ABCA4 partial coding sequence the 3′ AAV inverted terminal repeat.
22. The polynucleotide vector system of any one of embodiments 17-21, wherein the one or more substitutions are positioned in one or more putative in-frame stop codons.
23. The polynucleotide vector system of embodiment 22, wherein the substitutions are positioned in one or more putative in-frame stop codons in the AP head sequence.
24. The polynucleotide vector system of embodiment 23 wherein the substitutions are positioned in three putative in-frame stop codons in the AP intron sequence.
25. The polynucleotide vector system of any one of embodiments 1-24, wherein the promoter is selected from the group consisting of: a CMV promoter, an EF-1 alpha promoter, a cone arrestin promoter, a smCBA promoter, a human ABCA4 gene-derived promoter, a TαC gene-derived promoter, a rhodopsin promoter, a cGMP-phosphodiesterase β-subunit promoter, human or mouse rhodopsin promoter, a hGRK1 promoter, a rod specific IRBP promoter, a VMD2 promoter, and combinations thereof.
26. The polynucleotide vector system of embodiment 25, wherein the promoter is a CMV promoter.
27. The polynucleotide vector system of embodiment 25, wherein the promoter is a smCBA promoter.
28. The polynucleotide vector system of embodiment 25, wherein the promoter is a rhodopsin promoter.
29. The polynucleotide system of any one of embodiments 1-29, further comprising one or more nucleotide substitutions to remove one or more putative stop codons in a 3′ untranslated region between the partial coding sequence encoding the C-terminal part of the polypeptide and the 3′ AAV inverted terminal repeat of the second AAV vector polynucleotide.
30. The polynucleotide system of embodiment 29, wherein the one or more substitutions are located in one or more putative stop codons.
31. The polynucleotide vector system of any one of embodiments 1-30, wherein the second AAV vector polynucleotide is followed by a polyadenylation (pA) signal sequence.
32. The polynucleotide vector system of any one of embodiments 1-31, wherein the inverted terminal repeat at each end of the first AAV vector polynucleotide or the second AAV vector polynucleotide comprises a 5′ AAV ITR and a 3′ AAV ITR, and wherein the 5′ AAV ITR and the 3′ AAV ITR are ITRs from a single AAV serotype.
33. The polynucleotide vector system of any one of embodiments 1-32, wherein the inverted terminal repeat at each end of the first or the second AAV vector polynucleotide comprises a 5′ AAV ITR and a 3′ AAV ITR, and wherein the 5′ AAV ITR and the 3′ AAV ITR are ITRs from multiple AAV serotypes.
34. The polynucleotide vector system of any one of embodiments 1-28, wherein AAV inverted terminal repeats (ITRs) comprise ITRS from one or more AAV serotypes selected from the group consisting of: AAV serotype 2 (AAV2), AAV serotype 5 (AAV5), AAV serotype 7 (AAV7), AAV serotype 8 (AAV8), AAV serotype 44.9 (AAV44.9), AAV serotype 44.9 (E531D), and AAV serotype 44.9 (Y733F).
35. The polynucleotide vector system of embodiment 34, wherein the AAV serotype is AAV serotype 44.9 (E531D).
36. The polynucleotide vector system of embodiment 34 wherein the AAV serotype is AAV2.
37. A recombinant viral particle comprising the first AAV vector polynucleotide or the second AAV vector polynucleotide of any one of embodiments 1-36.
38. The recombinant viral particle of embodiment 37, comprising one or more tyrosine-to-phenylalanine (Y-F) mutations in a capsid protein of the virus or virion.
39. The recombinant viral particle of embodiment 37 or embodiment 38, wherein the recombinant viral particle comprises an AAV1, AAV2, AAV3, AAV4, AAV5, AAV6, AAV7, AAV8, AAV9, and/or AAV10 capsid.
40. The recombinant viral particle of any one of embodiments 37-39, wherein the recombinant viral particle comprises an AAV7m8, AAV-DJ, AAV2/2-MAX, AAVSHh10, AAVSHh10Y, AAV3b, AAVLK03, AAV8BP2, AAV1 (E531K), AAV6 (D532N), AAV6-3pmut, AAV2G9, AAV44.9, AAV44.9 (E531D), AAVrh.8, AAVrh.8R, and/or AAVAnc80 capsid.
41. The recombinant viral particle of embodiment 40, wherein the recombinant viral particle comprises an AAV44.9 (E531D) capsid.
42. An isolated host cell comprising the polynucleotide vector system of any one of embodiments 1-36 or the recombinant viral particle of any one of embodiments 37-41.
43. The isolated host cell of embodiment 42, wherein the cell is a photoreceptor cell, a cone cell, a rod cell, a retinal cell, a ganglion cell, a retinal pigment epithelium cell, a vestibular hair cell, an inner ear hair cell, an outer ear hair cell, or any combination thereof.
44. A method for treating or ameliorating a disease or condition in a human or animal, comprising administering to one or more cells of the human or animal, a polynucleotide vector system of any of embodiments 1-36 or the recombinant viral particle of any one of embodiments 37-41, wherein the ABCA4 or functional variant thereof provides for treatment or amelioration of a disease or condition and is expressed in the one or more cells.
45. The method of embodiment 44, wherein the disease or condition is Stargardt Disease.
46. The method of embodiment 44 or 45, wherein the ABCA4 polypeptide comprises the amino acid sequence of SEQ ID NO: 2, or a functional fragment thereof.
47. A method of administering the polynucleotide vector system of any one of embodiments 1-36 or the recombinant viral particle of any one of embodiments 37-41, whereby an amount of truncated ABCA4 protein produced by administration of the polynucleotide vector system is minimized.
48. A method of administering the polynucleotide vector system of any one of embodiments 1-36 or the recombinant viral particle of any one of embodiments 37-41, whereby cytotoxicity resulting from administration of the polynucleotide vector system is minimized.
49. The method of any one of embodiments 44-48, wherein administration of the polynucleotide vector system provides a partial or complete restoration of vision loss.
50. The method of any one of embodiments 44-49, wherein the polynucleotide vector system is administered by parenteral administration, intravenous administration, intramuscular administration, intraocular administration, intranasal administration, subretinal administration, round window injection, or during cochlear implant surgery.

What is claimed is:

1. A polynucleotide vector system for providing an ABCA4 gene, comprising
a. a first AAV vector polynucleotide comprising a first ABCA4 sequence of the ABCA4 gene, and
b. a second AAV vector polynucleotide comprising a second ABCA4 sequence of the ABCA4 gene,
wherein the ABCA4 gene encodes an ABCA4 protein at least 95% identical to SEQ ID NO: 2; the first ABCA4 sequence comprises exon 1 to exon 20 of the ABCA4 gene, and the second ABCA4 sequence comprises exon 21 to exon 50 of the ABCA4 gene.

2. The polynucleotide vector system of claim 1, wherein the first ABCA4 sequence and the second ABCA4 sequence do not overlap.

3. The polynucleotide vector system of claim 1, wherein the last about 5, 10, 15, 20, 25, 30, 35, 40, 45, or 50 nucleotides of the first ABCA4 sequence and the first about 5, 10, 15, 20, 25, 30, 35, 40, 45, or 50 nucleotides of the second ABCA4 sequence do not overlap.

4. The polynucleotide vector system of any one of claims 1 to 3, wherein the first ABCA4 sequence does not comprise any one of exons 21 to exons 50 of the ABCA4 gene, and the second ABCA4 sequence does not comprise any one of exons 1 to exons 20 of the ABCA4 gene.

5. The polynucleotide vector system any one of claims 1 to 4, wherein the ABAC4 gene is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 1.

6. The polynucleotide vector system of any one of claims 1 to 5, wherein the first ABCA4 sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 13.

7. The polynucleotide vector system of any one of claims 1 to 6, wherein the second ABCA4 sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 15.

8. The polynucleotide vector system of any one of claims 1 to 7, wherein the first AAV vector polynucleotide comprises a promoter upstream of the first ABCA4 sequence.

9. The polynucleotide vector system of claim 8, wherein the promoter is a smCBA promoter, CMV promoter, EF-1 alpha promoter, cone arrestin promoter, human ABCA4 promoter, TαC gene promoter, rhodopsin promoter, cGMP-phosphodiesterase β-subunit promoter, human rhodopsin promoter, mouse rhodopsin promoter, hGRK1 promoter, rod specific IRBP promoter, or VMD2 promoter.

10. The polynucleotide vector system of claim 8, wherein the promoter is a smCBA promoter.

11. The polynucleotide vector system of claim 8, wherein the promoter comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 23.

12. The polynucleotide vector system of any one of claims 1 to 11, wherein the first AAV vector polynucleotide comprises a splice donor site.

13. The polynucleotide vector system of claim 12, wherein the splice donor site is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 24.

14. The polynucleotide vector system of any one of claims 1 to 13, wherein the second AAV vector polynucleotide comprises a splice acceptor site.

15. The polynucleotide vector system of claim 14, wherein the splice acceptor site is at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 25.

16. The polynucleotide vector system of any one of claims 1 to 15, wherein the first AAV vector polynucleotide and/or the second AAV vector polynucleotide comprises an alkaline phosphatase (AP) head sequence.

17. The polynucleotide vector system of claim 16, wherein the first AAV vector polynucleotide comprises the AP head sequence, and the AP head sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 22.

18. The polynucleotide vector system of claim 16, wherein the second AAV vector polynucleotide comprises the AP head sequence, and the AP head sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 22.

19. The polynucleotide vector system of any one of claims 1-18, wherein the second AAV vector polynucleotide comprises a polyadenylation (pA) signal sequence.

20. The polynucleotide vector system of claim 19, wherein the pA signal sequence comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 21.

21. The polynucleotide vector system of any one of claims 1-20, wherein the first AAV vector polynucleotide and the second AAV vector polynucleotide each comprise a 5′ AAV ITR and a 3′ AAV ITR.

22. The polynucleotide vector system of claim 21, wherein the 5′ AAV ITR comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 26.

23. The polynucleotide vector system of claim 21 or claim 22, wherein the 3′ AAV ITR comprises a sequence at least 80%, 81%, 82%, 83%, 84%, 85%, 86%, 87%, 88%, 89%, 90%, 91%, 92%, 93%, 94%, 95%, 96%, 97%, 98%, or 99% identical to SEQ ID NO: 27.

24. The polynucleotide vector system of any one of claims 21 to 23, wherein the 5′ AAV ITR and the 3′ AAV ITR are of the AAV2 serotype.

25. A recombinant viral particle comprising the first AAV vector polynucleotide or the second AAV vector polynucleotide of any one of claims 1-24.

26. The recombinant viral particle of claim 25, wherein the recombinant viral particle comprises an AAV44.9 (E531D), AAV7m8, AAV-DJ, AAV2/2-MAX, AAVSHh10, AAVSHh10Y, AAV3b, AAVLK03, AAV8BP2, AAV1 (E531K), AAV6 (D532N), AAV6-3pmut, AAV2G9, AAV44.9, AAVrh.8, AAVrh.8R, or AAVAnc80 capsid.

27. The recombinant viral particle of claim 25, wherein the recombinant viral particle comprises an AAV44.9 (E531D) capsid.

28. An isolated host cell comprising the polynucleotide vector system of any one of claims 1-24 or the recombinant viral particle of any one of claims 25-27.

29. The isolated host cell of claim 28, wherein the cell is a photoreceptor cell, cone cell, rod cell, retinal cell, ganglion cell, retinal pigment epithelium cell, vestibular hair cell, inner ear hair cell, or outer ear hair cell.

30. A method for treating or ameliorating a disease or condition in a human or animal, comprising administering to one or more cells of the human or animal, a polynucleotide vector system of any of claims 1-24 or the recombinant viral particle of any one of claims 25-27, wherein expression of the ABCA4 gene treats or ameliorates the disease or condition and is expressed in the one or more cells.

31. The method of claim 30, wherein the disease or condition is Stargardt Disease.

32. The method of claim 30 or claim 31, wherein the treating provides a partial or complete restoration of vision loss.

33. The method of any one of claims 30-32, wherein the polynucleotide vector system is administered by parenteral administration, intravenous administration, intramuscular administration, intraocular administration, intranasal administration, subretinal administration, round window injection, or during cochlear implant surgery.


SEQUENCE LISTING (29 sequences)

SEQ ID NO: 1 - DNA, 6819 bp, Homo sapiens
  source 1..6819: mol_type = other DNA
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggac

SEQ ID NO: 2 - protein, 2273 aa, Homo sapiens [sequence omitted]

SEQ ID NO: 3 - DNA, 4604 bp, synthetic construct
  source 1..4604: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctggcgcgcccaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttggtaagtatcaaggttacaagacaggttaacggagaccaattgaaactgggcttgtcgagacagagaagactcttgcgtttcagcgctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttcgtcgacggatccgcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 4 - DNA, 4771 bp, synthetic construct
  source 1..4771: mol_type = other DNA
ggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttcgtcgactgatcctaggcacctattggtcttactgacatccactttgcctttctctccacaggtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactacccttacgatgtaccggattacgcatgagcggccgcttgccagccatctgttgtttgcccctcccccgtgccttccttgaccctggaaggtgccactcccactgtcctttcctaataaaatgaggaaattgcatcgcattgtctgagtaggtgtcattctattctggggggtgggtacctagcactagggctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacc

SEQ ID NO: 5 - DNA, 4736 bp, synthetic construct
  source 1..4736: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctggcgcgcccaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacgtaagtatcaaggttacaagacaggttaacggagaccaattgaaactgggcttgtcgagacagagaagactcttgcgtttcagcgctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttcgtcgacggatccgcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 6 - DNA, 4638 bp, synthetic construct
  source 1..4638: mol_type = other DNA
ggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttcgtcgactgatcctaggcacctattggtcttactgacatccactttgcctttctctccacagctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactacccttacgatgtaccggattacgcatgagcggccgcttgccagccatctgttgtttgcccctcccccgtgccttccttgaccctggaaggtgccactcccactgtcctttcctaataaaatgaggaaattgcatcgcattgtctgagtaggtgtcattctattctggggggtgggtacctagcactagggctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacc

SEQ ID NO: 7 - DNA, 4874 bp, synthetic construct
  source 1..4874: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctggcgcgcccaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtaagtatcaaggttacaagacaggttggcggagaccaattgaaactgggcttgtcgagacagagaagactcttgcgtttcacgctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttcgtcgacggatccgcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 8 - DNA, 4499 bp, synthetic construct
  source 1..4499: mol_type = other DNA
ggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctcagatctgaattcggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttcgtcgactgatcctaggcacctattggtcttactgacatccactttgcctttctctccacaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactacccttacgatgtaccggattacgcatgagcggccgcttgccagccatctgttgtttgcccctcccccgtgccttccttgaccctggaaggtgccactcccactgtcctttcctaataaaatgaggaaattgcatcgcattgtctgagtaggtgtcattctattctggggggtgggtacctagcactagggctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacc

SEQ ID NO: 9 - DNA, 2919 bp, synthetic construct
  source 1..2919: mol_type = other DNA
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttg

SEQ ID NO: 10 - protein, 973 aa, synthetic construct
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTL

SEQ ID NO: 11 - DNA, 3259 bp, synthetic construct
  source 1..3259: mol_type = other DNA
gtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggac

SEQ ID NO: 12 - protein, 1300 aa, synthetic construct
SILTGLLPPTSGTVLVGGRDIETSLDAVRQSLGMCPQHNILFHHLTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 13 - DNA, 3051 bp, synthetic construct
  source 1..3051: mol_type = other DNA
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccac

SEQ ID NO: 14 - protein, 1017 aa, synthetic construct
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHH

SEQ ID NO: 15 - DNA, 3768 bp, synthetic construct
  source 1..3768: mol_type = other DNA
ctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggac

SEQ ID NO: 16 - protein, 1256 aa, synthetic construct
LTVAEHMLFYAQLKGKSQEEAQLEMEAMLEDTGLHHKRNEEAQDLSGGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 17 - DNA, 3190 bp, synthetic construct
  source 1..3190: mol_type = other DNA
atgggcttcgtgagacagatacagcttttgctctggaagaactggaccctgcggaaaaggcaaaagattcgctttgtggtggaactcgtgtggcctttatctttatttctggtcttgatctggttaaggaatgccaacccactctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcag

SEQ ID NO: 18 - protein, 1063 aa, synthetic construct
MGFVRQIQLLLWKNWTLRKRQKIRFVVELVWPLSLFLVLIWLRNANPLYSHHECHFPNKAMPSAGMLPWLQGIFCNVNNPCFQSPTPGESPGIVSNYNNSILARVYRDFQELLMNAPESQHLGRIWTELHILSQFMDTLRTHPERIAGRGIRIRDILKDEETLTLFLIKNIGLSDSVVYLLINSQVRPEQFAHGVPDLALKDIACSEALLERFIIFSQRRGAKTVRYALCSLSQGTLQWIEDTLYANVDFFKLFRVLPTLLDSRSQGINLRSWGGILSDMSPRIQEFIHRPSMQDLLWVTRPLMQNGGPETFTKLMGILSDLLCGYPEGGGSRVLSFNWYEDNNYKAFLGIDSTRKDPIYSYDRRTTSFCNALIQSLESNPLTKIAWRAAKPLLMGKILYTPDSPAARRILKNANSTFEELEHVRKLVKAWEEVGPQIWYFFDNSTQMNMIRDTLGNPTVKDFLNRQLGEEGITAEAILNFLYKGPRESQADDMANFDWRDIFNITDRTLRLVNQYLECLVLDKFESYNDETQLTQRALSLLEENMFWAGVVFPDMYPWTSSLPPHVKYKIRMDIDVVEKTNKIKDRYWDSGPRADPVEDFRYIWGGFAYLQDMVEQGITRSQVQAEAPVGIYLQQMPYPCFVDDSFMIILNRCFPIFMVLAWIYSVSMTVKSIVLEKELRLKETLKNQGVSNAVIWCTWFLDSFSIMSMSIFLLTIFIMHGRILHYSDPFILFLFLLAFSTATIMLCFLLSTFFSKASLAAACSGVIYFTLYLPHILCFAWQDRMTAELKKAVSLLSPVAFGFGTEYLVRFEEQGLGLQWSNIGNSPTEGDEFSFLLSMQMMLLDAAVYGLLAWYLDQVFPGDYGTPLPWYFLLQESYWLGGEGCSTREERALEKTEPLTEETEDPEHPEGIHDSFFEREHPGWVPGVCVKNLVKIFEPCGRPAVDRLNITFYENQITAFLGHNGAGKTTTLSILTGLLPPTSGTVLVGGRDIETSLDAVRQSLGMCPQHNILFHHLTVAEHMLFYAQLKGKSQEEAQLEMEAMLEDTGLHHKRNEEAQDLS

SEQ ID NO: 19 - DNA, 3628 bp, synthetic construct
  source 1..3628: mol_type = other DNA
tggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggac

SEQ ID NO: 20 - protein, 1210 aa, synthetic construct
GGMQRKLSVAIAFVGDAKVVILDEPTSGVDPYSRRSIWDLLLKYRSGRTIIMSTHHMDEADLLGDRIAIIAQGRLYCSGTPLFLKNCFGTGLYLTLVRKMKNIQSQRKGSEGTCSCSSKGFSTTCPAHVDDLTPEQVLDGDVNELMDVVLHHVPEAKLVECIGQELIFLLPNKNFKHRAYASLFRELEETLADLGLSSFGISDTPLEEIFLKVTEDSDSGPLFAGGAQQKRENVNPRHPCLGPREKAGQTPQDSNVCSPGAPAAHPEGQPPPEPECPGPQLNTGTQLVLQHVQALLVKRFQHTIRSHKDFLAQIVLPATFVFLALMLSIVIPPFGEYPALTLHPWIYGQQYTFFSMDEPGSEQFTVLADVLLNKPGFGNRCLKEGWLPEYPCGNSTPWKTPSVSPNITQLFQKQKWTQVNPSPSCRCSTREKLTMLPECPEGAGGLPPPQRTQRSTEILQDLTDRNISDFLVKTYPALIRSSLKSKFWVNEQRYGGISIGGKLPVVPITGEALVGFLSDLGRIMNVSGGPITREASKEIPDFLKHLETEDNIKVWFNNKGWHALVSFLNVAHNAILRASLPKDRSPEEYGITVISQPLNLTKEQLSEITVLTTSVDAVVAICVIFSMSFVPASFVLYLIQERVNKSKHLQFISGVSPTTYWVTNFLWDIMNYSVSAGLVVGIFIGFQKKAYTSPENLPALVALLLLYGWAVIPMMYPASFLFDVPSTAYVALSCANLFIGINSSAITFILELFENNRTLLRFNAVLRKLLIVFPHFCLGRGLIDLALSQAVTDVYARFGEEHSANPFHWDLIGKNLFAMVVEGVVYFLLTLLVQRHFFLSQWIAEPTKEPIVDEDDDVAEERQRIITGGNKTDILRLHELTKIYPGTSSPAVDRLCVGVRPGECFGLLGVNGAGKTTTFKMLTGDTTVTSGDATVAGKSILTNISEVHQNMGYCPQFDAIDELLTGREHLYLYARLRGVPAEEIEKVANWSIKSLGLTVYADCLAGTYSGGNKRKLSTAIALIGCPPLVLLDEPTTGMDPQARRMLWNVIVSIIREGRAVVLTSHSMEECEALCTRLAIMVKGAFRCMGTIQHLKSKFGDGYIVTMKIKSPKDDLLPDLNPVEQFFQGNFPGSVQRERHYNMLQFQVSSSSLARIFQLLLSHKDSLLIEEYSVTQTTLDQVFVNFAKQQTESHDLPLHPRAAGASRQAQD

SEQ ID NO: 21 - DNA, 139 bp, synthetic construct
  source 1..139: mol_type = other DNA
ttgccagccatctgttgtttgcccctcccccgtgccttccttgaccctggaaggtgccactcccactgtcctttcctaataaaatgaggaaattgcatcgcattgtctgagtaggtgtcattctattctggggggtggg

SEQ ID NO: 22 - DNA, 287 bp, synthetic construct
  source 1..287: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttc

SEQ ID NO: 23 - DNA, 950 bp, synthetic construct
  source 1..950: mol_type = other DNA
ggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaatt

SEQ ID NO: 24 - DNA, 90 bp, synthetic construct
  source 1..90: mol_type = other DNA
gtaagtatcaaggttacaagacaggttaacggagaccaattgaaactgggcttgtcgagacagagaagactcttgcgtttcagcgctagc

SEQ ID NO: 25 - DNA, 49 bp, synthetic construct
  source 1..49: mol_type = other DNA
taggcacctattggtcttactgacatccactttgcctttctctccacag

SEQ ID NO: 26 - DNA, 145 bp, synthetic construct
  source 1..145: mol_type = other DNA
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 27 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 28 - protein, 736 aa, synthetic construct
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMASQARNWVPGPSYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 29 - DNA, 772 bp, synthetic construct
  source 1..772: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttcgtcgacggatccgcatgctggggagagatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaacagatctgaattcggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttc